SEQUENCE LISTING (18 sequences)

SEQ ID NO: 1 - DNA, 22 bp, synthetic construct
  source 1..22: mol_type = other DNA
cgggcagtgtttgtagacttgg

SEQ ID NO: 2 - DNA, 22 bp, synthetic construct
  source 1..22: mol_type = other DNA
ctccttgccaatggtgtagtgc

SEQ ID NO: 3 - DNA, 20 bp, synthetic construct
  source 1..20: mol_type = other DNA
gcgagatgtacgaagacgac

SEQ ID NO: 4 - DNA, 20 bp, synthetic construct
  source 1..20: mol_type = other DNA
tttagacactgctggcttcg

SEQ ID NO: 5 - DNA, 21 bp, synthetic construct
  source 1..21: mol_type = other DNA
cctctttccaaagtatagcac

SEQ ID NO: 6 - DNA, 22 bp, synthetic construct
  source 1..22: mol_type = other DNA
ttactggcattcatgtcatttc

SEQ ID NO: 7 - DNA, 22 bp, synthetic construct
  source 1..22: mol_type = other DNA
tgctcagacaggatgtgagttg

SEQ ID NO: 8 - DNA, 21 bp, synthetic construct
  source 1..21: mol_type = other DNA
aattacagcaagcctggaacc

SEQ ID NO: 9 - DNA, 25 bp, synthetic construct
  source 1..25: mol_type = other DNA
tatagctcagatcattgtcacagtc

SEQ ID NO: 10 - DNA, 21 bp, synthetic construct
  source 1..21: mol_type = other DNA
gttggtcgtcaggaagaagag

SEQ ID NO: 11 - DNA, 20 bp, synthetic construct
  source 1..20: mol_type = other DNA
accgcatctctacattcaag

SEQ ID NO: 12 - DNA, 18 bp, synthetic construct
  source 1..18: mol_type = other DNA
caagtctggctcgttctc

SEQ ID NO: 13 - DNA, 25 bp, synthetic construct
  source 1..25: mol_type = other DNA
caaatgattccaagcataaaagaac

SEQ ID NO: 14 - DNA, 25 bp, synthetic construct
  source 1..25: mol_type = other DNA
ggtaaatctacatcctcatcaccac

SEQ ID NO: 15 - DNA, 19 bp, synthetic construct
  source 1..19: mol_type = other DNA
ggatgctggaggtctgcga

SEQ ID NO: 16 - DNA, 20 bp, synthetic construct
  source 1..20: mol_type = other DNA
agaggccacgaacatgcaag

SEQ ID NO: 17 - DNA, 19 bp, synthetic construct
  source 1..19: mol_type = other DNA
acagtccagccgcatcttc

SEQ ID NO: 18 - DNA, 19 bp, synthetic construct
  source 1..19: mol_type = other DNA
gcccaatacgaccaaatcc

(19) United States
(12) Patent Application Publication
(10) Pub. No.: US 2024/0240256 A1
(43) Pub. Date: Jul. 18, 2024

(54) MOLECULAR ANALYSIS OF EXTRACELLULAR VESICLES (EVs) FOR THE PREDICTION AND MONITORING OF DRUG RESISTANCE IN CANCER

(71) Applicant: The General Hospital Corporation, Boston, MA (US)

(72) Inventors: Hyungsoon Im, Peabody, MA (US); Ursula A. Winter, Boston, MA (US)

(21) Appl. No.: 18/410,620

(22) Filed: Jan. 11, 2024

Related U.S. Application Data
(60) Provisional application No. 63/500,317, filed on May 5, 2023.
(60) Provisional application No. 63/479,624, filed on Jan. 12, 2023.

(51) Int. Cl. C12Q 1/6886; C12Q 1/6851; G01N 33/574

(52) U.S. Cl. CPC C12Q 1/6886; C12Q 1/6851; G01N 33/57484; C12Q 2600/106; C12Q 2600/158; G01N 2333/4739; G01N 2333/70596

(57) ABSTRACT

Provided herein are methods of predicting a subject's response to chemotherapeutic drugs. Additionally provided are methods of monitoring a subject's response to a chemotherapeutic drug over time, e.g., monitoring for chemotherapy resistance. The methods include enriching a sample for tumor-derived extracellular vesicles (tEVs) and determining the relative level of a subject's drug-resistance biomarkers compared to a subject's drug-resistance biomarkers at a previous time point.

### CLAIM OF PRIORITY

This application claims the benefit of U.S. Provisional Application Ser. No. 63/479,624, filed on Jan. 12, 2023, and Ser. No. 63/500,317, filed on May 5, 2023. The entire contents of the foregoing are incorporated herein by reference.

### SEQUENCE LISTING

This application contains a Sequence Listing that has been submitted electronically as an XML file named “29539-0722001_SL_ST26.XML.” The XML file, created on Jan. 10, 2024, is 16,999 bytes in size. The material in the XML file is hereby incorporated by reference in its entirety.

### TECHNICAL FIELD

Assessment of molecular profiles of specific biomarkers in EVs as a non-invasive biosignature and prediction of pharmacological response and clinical outcome in patients with cancer.

### BACKGROUND

Cancer remains a global health challenge despite technological advancements. Resistance to therapies and disease recurrence hinder treatment progress. New treatments are needed as many cancers develop resistance to current therapies.

### SUMMARY

Provided herein are methods for predicting chemotherapy resistance in a subject with cancer that include providing a sample from the subject; isolating, detecting, or enriching tumor-derived extracellular vesicles (tEVs) from the sample, preferably wherein the tEVs are labeled with antibodies or antigen binding portions thereof that bind to tumor marker(s) and antibodies or antigen binding portions thereof that bind to chemotherapy resistance biomarker(s); determining the counts of tEVs positive for tumor marker(s) and chemotherapy resistance biomarker(s), or determining the intensity levels of tumor marker(s) and chemotherapy resistance biomarker(s) expressed in tEVs; and comparing the counts of tEVs or the marker intensity levels determined in the previous step to a reference level that represents the subject's cancer response to a chemotherapy, wherein the counts of tEVs or the marker intensity levels determined the previous step that differ from the reference levels indicate whether the subject's cancer is resistant or sensitive to the chemotherapy.

In some embodiments, the determining step of the above method further comprises using a plasmon-enhanced EV detection method in step. In some embodiments, the antibodies or antigen binding portions thereof that bind to the EV tumor marker(s) further comprise fluorescent dyes. In some embodiments, the antibodies or antigen binding portions thereof that bind to the EV tumor markers comprise one or more antibodies or antigen binding portions thereof that bind to EpCAM, EGFR, MUC1, and/or HER2. In some embodiments, the EVs are detected using a protein-reactive TFP dye that comprises fluorescent dye. In some embodiments, the chemotherapy resistance biomarkers comprise protein or RNA. In some embodiments, the chemotherapy resistance biomarkers are P-gp and survivin. In some embodiments, the quantification of EV tumor markers and chemotherapy resistance biomarkers comprises expression, concentration, intensity, or colocalization. In some embodiments, the quantification of expression, concentration, intensity, or colocalization are analyzed using multichannel fluorescence imaging in a single EV. In some embodiments, the cancer comprises breast cancer, ovarian cancer, and non-small cell lung cancer. In some embodiments, the sample obtained from the subject with cancer comprises tumor cells or plasma.

Also provided herein are methods for monitoring drug-resistance longitudinally in a subject having cancer that include: providing a sample from the subject, where the sample is acquired from the same subject at multiple time points during treatment with a chemotherapy; isolating tumor-derived EVs (tEVs) from the sample, wherein the tEVs are further double labeled with EV tumor marker(s) and drug-resistance biomarker(s); determining colocalization of EV tumor marker(s) and drug-resistance biomarker(s); and detecting changes in colocalization of EV tumor marker(s) and drug-resistance biomarker(s) before and after the chemotherapy treatment, thereby determining drug-resistance in the subject based on the changes of the quantitative colocalization of EV marker(s) and drug-resistance biomarker(s) before and after chemotherapy treatment.

Provided herein are methods for monitoring drug-resistance in a subject with cancer over time that include: isolating tumor extracellular vesicles (tEVs) in a first sample obtained from a subject at a first time point, wherein isolating tEVs comprises: applying the first sample to a functionalized substrate to capture extracellular vesicles (EVs); labeling the EVs with an antibody or antigen binding portions thereof that bind to a previously selected EV tumor marker(s); and labeling the EVs with an antibody or antigen binding portions thereof that bind to previously a selected drug-resistance biomarker(s); determining a count of tEVs positive for tumor marker(s) and chemotherapy resistance biomarker(s) or a level of drug-resistance biomarker(s) in the tEVs at a first time point; administering one or more doses of a chemotherapy drug; isolating tEVs in a second sample from the subject obtained at a second time point, wherein the isolating tEVs comprises: applying the second sample to a functionalized substrate to capture EVs; labeling the EVs with an antibody or antigen binding portions thereof that bind to the previously selected EV tumor marker(s); and labeling the EVs with an antibody or antigen binding portions thereof that bind to previously a selected drug-resistance biomarker(s); determining the count of tEVs positive for tumor marker(s) and chemotherapy resistance biomarker(s) or the level of drug-resistance biomarker(s) in the tEVs at a second time point; and administering one or more additional doses of the chemotherapy drug to the subject if the count of tEVs positive for tumor marker(s) and chemotherapy resistance biomarker(s) or the relative level of drug-resistance biomarker(s) have not increased from the first time point to the second time point.

In some embodiments, the above method further comprises using a plasmon-enhanced EV detection method. In some embodiments, the tEVs are selected by a marker panel comprising EpCAM, EGFR, MUC1, and/or HER2. In some embodiments, the antibodies or antigen binding portions thereof that bind to EV tumor markers further comprise fluorescent dyes. In some embodiments, the drug-resistance biomarkers comprise protein or RNA. In some embodiments, the drug-resistance biomarkers are P-gp and survivin. In some embodiments, the quantification of the colocalization of EV marker(s) and drug-resistance biomarker(s) are analyzed using multichannel fluorescence imaging in a single EV. In some embodiments, the cancer comprises breast cancer, ovarian cancer, or non-small cell lung cancer. In some embodiments, the sample obtained from the subject with cancer comprises plasma. In some embodiments, the method can identify drug-resistance prior to an observable increase in size of the subject's tumor. In some embodiments, the method further comprises recommending, prescribing and/or administering a therapeutically effective amount of a chemotherapy to a subject.

Also provided herein are methods for monitoring drug-resistance in a subject with cancer over time that include: isolating tumor extracellular vesicles (tEVs) in a first sample obtained from a subject at a first time point, wherein isolating tEVs comprises applying the sample to a surface comprising capture antibodies or antigen binding portions thereof that bind to previously selected EV tumor marker(s) and labeling the captured tEVs with antibodies or antigen binding portions thereof that bind to previously a selected drug-resistance biomarker(s); determining a count of tEVs positive for tumor marker(s) and chemotherapy resistance biomarker(s) or a level of drug-resistance biomarker(s) in the tEVs at a first time point; administering one or more doses of a chemotherapy drug; isolating tEVs in a second sample from the subject obtained at a second time point after administration of the one or more doses of the chemotherapy drug, wherein isolating the tEVs comprises applying a second sample to a surface comprising capture antibodies or antigen binding portions thereof that bind to the previously selected EV tumor marker(s) and labeling the captured tEVs with the antibodies or antigen binding portions thereof that bind to previously a selected drug-resistance biomarker(s); determining a count of tEVs positive for tumor marker(s) and chemotherapy resistance biomarker(s) or a level of drug-resistance biomarker(s) in the tEVs at the second time point; and administering one or more additional doses of the chemotherapy drug to the subject if the count of tEVs positive for tumor marker(s) and chemotherapy resistance biomarker(s) or the relative level of drug-resistance biomarker(s) have not increased from the first time point to the second time point.

In some embodiments, the capture antibodies or antigen binding portions thereof that bind to the EV tumor markers comprise one or more antibodies or antigen binding portions thereof that bind to EpCAM, EGFR, MUC1, and/or HER2. In some embodiments, the drug-resistance biomarker(s) comprises P-gp and/or survivin. In some embodiments, the cancer is breast cancer. In some embodiments, the chemotherapy is paclitaxel. In some embodiments, the first sample and/or the second sample comprises plasma. In some embodiments, the efficacy of predicting chemotherapy resistance over time in a subject with cancer is at least 95%.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1G show the effect of paclitaxel in vitro by growth inhibition assay performed on various cell lines including (FIG. 1A) HCC1954, (FIG. 1B) BT-474, (FIG. 1C) MCF7, (FIG. 1D) MDA-MB-231, (FIG. 1E) HCC1937 and (FIG. 1F) HCC1954-REPX cells. Symbols represent % of cell viability as compared to untreated control cells, expressed as means (SEM) from independent experiments with three replicates per drug concentration. In FIG. 1G, Bar chart shows the estimated median lethal dose (LC50) of paclitaxel's toxicity on the six tested cell lines, presented in terms of concentration (μM).

FIGS. 2A-2G show nanoparticle tracking analysis (NTA) of EVs. The size repartition was evaluated for the EVs secreted by (FIG. 2A) HS 371 T, (FIG. 2B) HCC1954, (FIG. 2C) BT-474, (FIG. 2D) MCF7, (FIG. 2E) MDA-MB-231, (FIG. 2F) HCC1937, and (FIG. 2G) HCC1954-REPX cell lines. The majority of EV populations fall within the size range of 85.7 to 179.1 nm, a commonly given size range to small EVs.

FIGS. 3A-3K show the qPCR analysis of gene expression profiles. Molecular profiling of cell lines and their corresponding-derived EVs were assessed for (FIG. 3A) ABCB1; (FIG. 3B, no EV data for ABCG2) ABCG2; (FIG. 3C) TUBA1A; (FIG. 3D) TUBB3; (FIG. 3E) CIC-3; (FIG. 3F) survivin; (FIG. 3G) CCND1; (FIG. 3H) PTGES3; (FIG. 3I, no EV data for miR-421) miR-421; (FIG. 3J, no EV data for miR-9) miR-9 and (FIG. 3K) miR-21. RT-qPCR data are shown as a mean of the logarithm of 2-ΔΔCT values for the relative expression of genes of interest (n=11). Error bars represent the standard deviation between biological replicates.

FIGS. 4A-4B show protein biomarkers in cell line and cell line-derived EVs. Proteome analysis was conducted on the tested cell lines (FIG. 4A) and their respective EVs (FIG. 4B). As shown in FIG. 4A, following fixation and permeabilization, cells were stained using primary antibodies for P-gp, survivin, Cyclin D1, and TSG101 (used as an internal control) or an isotype control. As shown in FIG. 4B, EVs derived from cell lines were captured on 5 μm beads and subsequently stained using the same antibodies as used in FIG. 4A. The heatmaps display the mean fluorescence intensity (MFI) of each sample normalized with the corresponding fluorescent signal of the isotype control antibody (MFITarget—MFIIsotype).

FIGS. 5A-5G show single EV analysis for P-gp and survivin on NPOP substrate. EV was labeled in solution with a protein-reactive TFP dye (Alexa Fluor™ 555) and biomarker-specific fluorescent antibodies for P-gp and survivin or isotype control. Following TFP labeling, the EVs were plated onto gold nanoplasmonic chips for plasmon-enhanced imaging by fluorescence microscopy. FIG. 5A shows an overview of multi-channel single EV analysis. NPOP substrate was functionalized by SH-PEG-COOH (1.0 kDa), and Alexa Fluor™ 555-labeled EVs were captured by EDC/NHS activation. FIGS. 5B-5D depict P-gp positive EVs as identified by biomarker specific antibodies measured in Alexa Fluor™ 488 channel; P-gp (FIG. 5B), IgG isotype control (FIG. 5C), and percentage (%) colocalization of P-gp minus IgG control (FIG. 5D), which identifies the colocalization of the biomarker specific antibodies on the same EV. FIGS. 5E-5G depict survivin positive EVs as identified by biomarker specific antibodies measured in Alexa Fluor™ 647channel; survivin (FIG. 5E), IgG isotype control (FIG. 5F), and percentage (%) colocalization of survivin minus IgG (FIG. 5G), which identifies the colocalization of the biomarker specific antibodies on the same EV.

FIGS. 6A-6C show multiplexed single tumor-derived EV (tEV) drug-resistant marker detection in single tumor-derived EV (tEV) on NPOP substrate. FIG. 6A shows the representative images of selective capture of tEVs using QUAD markers (MUC1, HER2, EGFR, and EpCAM). In FIG. 6B, the bar graph depicts the number of positive tEVs identified in the channel of Alexa Fluor™ 555-labeled QUAD marker antibody mix. In FIG. 6C, the bar graph depicts the number of positive tEVs identified in the colocalization of Alexa Fluor™ 555-labeled QUAD marker antibody mix and Alexa Fluor™ 647-labeled Pgp/survivin antibody mix for each patient at two-time points: before chemotherapy (T1) and before surgery (after neoadjuvant chemotherapy, T2) for 2 years.

FIGS. 7A-7D show the analysis of changes of tEVs and PgP/survivin-positive tEV counts before and after receiving paclitaxel treatment. EVs from two plasma samples obtained before and after paclitaxel neoadjuvant therapy were processed. FIG. 7A shows the relative and FIG. 7B shows the percentage change of QUAD-positive EVs. FIG. 7C shows the relative and FIG. 7D shows the percentage change of PgP/survivin-positive tEVs. Patients P2, P3, P5, P6, P7, P10, P11, P14, P16, P17, P18, and P20 demonstrated pathological complete response from neoadjuvant therapy with paclitaxel, and patients P1, P4, P8, P9, P12, P13, P15, P19, P21, and P22 did not show pathological complete response to paclitaxel neoadjuvant therapy.

FIGS. 8A-8F show a summary of up-regulated genes in paclitaxel-resistant cancer cell lines (HCC1954 REPX, BT474 RETX) compared to wild-type cancer cell lines (HCC1954, BT474). Specifically, FIG. 8A shows MDR1 is upregulated in HCC1954 REPX and BT474 RETX cells compared to Hs371T, HCC1954 and BT474 cells. FIG. 8B shows survivin is upregulated in Hs37IT, HCC1954, HCC1954 REPX, BT474, and BT474 REPX cells. FIG. 8C shows ABCG2 is upregulated in HCC1954 REPX and BT474 RETX cells compared to Hs371T, HCC1954 and BT474 cells. FIG. 8D shows miRNA9 shows highest upregulation in paclitaxel-resistant HCC1954 REPX. FIG. 8E shows miRNA421 shows highest upregulation in paclitaxel-resistant BT474 REPX. FIG. 8F shows miRNA21 shows highest upregulation in paclitaxel-resistant HCC1954 REPX and BT474 REPX cell lines.

FIGS. 9A-9D show a summary of down-regulated genes in paclitaxel-resistant cell lines (HCC1954 REPX, BT474 RETX) compared to wild-types (HCC1954, BT474). Specifically, FIG. 9A shows downregulation of TUB1A1 across all cell lines tested. FIG. 9B shows downregulation of TUBB across all cell lines tested. FIG. 9C shows downregulation of PTGES3 across all cell lines tested. FIG. 9D shows downregulation of CYCLIN D across all cell lines tested, with greatest downregulation in Hs371T and HCC1954 REPX cell lines.

FIGS. 10A-10E show single EV analysis by NPOP substrates. PgP- and survivin-positive EV counts were significantly increased in those from paclitaxel-resistant cell lines. FIG. 10A shows EV count across cell lines tested. FIG. 10B shows MDR1 EV count across cell lines tested. FIG. 10C shows EV count for IgG controls. FIG. 10D shows colocalization of PgP-positive EVs and QUAD markers (MUC1, HER2, EGFR, and EpCAM) in paclitaxel-resistant cell lines MDA-MB-231, HCC1937, and HCC1954 REPX. FIG. 10E shows colocalization of survivin-positive EVs and QUAD markers (MUC1, HER2, EGFR, and EpCAM) in paclitaxel-resistant cell lines MDA-MB-231, HCC1937, and HCC1954 REPX.

### DETAILED DESCRIPTION

As shown herein, assessment of molecular profiles of specific biomarkers in EVs can be used as a non-invasive biosignature of pharmacological response and clinical outcome in patients with cancer.

Provided herein are methods of predicting response to a chemotherapeutic drug (e.g., paclitaxel (px)) in a subject (e.g., a subject who has breast cancer) by determining levels of drug-resistant biomarker(s) (e.g., p-glycoprotein (P-gP) and/or survivin) in a sample comprising tumor cells from the subject; optionally the methods comprising isolating EVs from the sample before determining the levels. P-gp and/or survivin protein or mRNA levels of the sample can be compared to a reference level, and levels below a reference level for drug-resistant biomarkers (e.g., P-gp and/or survivin biomarkers) indicate that the tumor is sensitive to px. In some embodiments, the levels are analyzed by plasmon-enhanced single EV assay⁹. In some embodiments, the methods further include recommending, prescribing and/or administering a therapeutically effective amount of px to the subject.

In some embodiments, the method includes assaying drug-resistant biomarker(s) (e.g., Pg-p/survivin) levels over time in a subject with cancer, wherein an increase in the drug-resistant biomarker(s) (e.g., P-gp and/or survivin biomarkers), indicates that the subject or tumor is developing resistance to a chemotherapeutic drug (e.g., px). In some embodiments, the methods further include treating with chemotherapy if the drug-resistant biomarker(s) (e.g. P-gp and/or survivin levels) increase above a threshold or increase relative to a drug-resistant biomarker level from the subject at an earlier time point.

Method of Monitoring Drug Resistance Using tEVs

The present methods can include isolating particular EV populations (e.g., tumor-derived EVs) in a subject being treated for cancer and measuring the tEVs expression of drug-resistant biomarkers over time and further administering chemotherapy informed by the relative levels of drug-resistance biomarker-positive (e.g., P-gp and/or survivin-positive) tEVs over time.

A subject can be an individual (e.g., a mammal such as a human) having or suspected of having cancer. In some embodiments, the subject can be receiving chemotherapy, and/or another type of cancer treatment (e.g., radiation, surgery). A sample can be obtained from the subject. A sample can mean any sample, including, but not limited to cells, lysed cells, cellular extracts, nuclear extracts, extracellular fluid, media in which cells (e.g., cancer cells from the subject) are cultured, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears and prostatic fluid. In some embodiments, a sample is obtained from a subject at multiple time points.

The sample from the subject can be enriched for EVs, e.g., based on the presence of EV tumor markers. In some embodiments, the methods can include using antibodies or antigen binding portions thereof that bind to selected EV tumor markers corresponding to a particular type of cancer in order to identify or enrich tEVs for further analysis. For example, the antibodies can be capture antibodies that are attached to a substrate (e.g., a plate, well, or beads). A sample from a subject, e.g., a sample comprising a population of EVs (optionally EVs obtained from a biofluid such as blood, serum, or plasma) can then be applied to the substrate, wherein the antibodies or antigen binding portions thereof that bind to the selected EV tumor markers capture and enrich the EV population for tEVs having the specified EV tumor markers. Drug-resistance biomarkers can then be evaluated in the tEVs, e.g., optionally using antibodies that bind the resistance biomarkers, to determine a level of drug-resistance biomarker for that sample and subject.

In some embodiments, the antibodies or antigen binding portions thereof that bind to the selected EV tumor markers can be applied to a sample, wherein the sample has been previously enriched for EVs. One method for enriching a sample for EVs can include subjecting the sample to a plasmon-enhanced EV assay. For example, the sample can be applied to a 3D plasmonic nanostructure composed of spherical Au nanoparticles on 3D Au nanopillars (NPOP) substrate, wherein EVs are captured by the NPOP substrate. See Park, et al. Self-assembly of nanoparticle-spiked pillar arrays for plasmonic biosensing, Adv. Funct. Mater., 1904257 (2019). In some embodiments, the antibodies or antigen binding portions thereof that bind to the selected EV tumor markers can be applied as free antibodies to the EV sample, wherein the antibodies or antigen binding portions thereof that bind to the selected EV tumor markers can be labeled (e.g., fluorescently labeled) or wherein the antibodies or antigen binding portions thereof that bind to the selected EV tumor markers can be detected with a secondary antibody. In some embodiments the antibodies or antigen binding portions thereof that bind to the selected EV tumor markers can be applied before, after, concurrently with the antibodies or antigen binding portions thereof that bind to selected drug-resistance biomarker(s).

In some embodiments, EVs from a sample can be enriched using a plasmon-enhanced EV capture method. In some embodiments, the plasmon-enhanced EV capture method includes EV capture using any substrate, e.g., plain substrate, nanostructures, beads, or other materials. In some embodiments, the plasmon-enhanced EV capture method includes EV capture using an NPOP substrate, wherein in some embodiments the NPOP substrate can be constructed and/or functionalized according to the methods described in the examples. EVs that have been enriched by isolation on an NPOP substrate can be probed for expression of EV tumor marker(s) and/or drug-resistance biomarker(s). Antibodies to EV tumor markers can be applied to the EV-enriched sample, wherein the antibodies to EV tumor marker(s) can be labeled (e.g., fluorescently labeled) or wherein secondary antibodies can be used to detect the antibodies to EV tumor marker(s). Antibodies to drug-resistance biomarker(s) can be applied to the EV-enriched sample, wherein the antibodies to the drug-resistance biomarker(s) can be labeled (e.g., fluorescently labeled) or wherein secondary antibodies can be used to detect the antibodies to drug-resistance biomarker(s). In some embodiments, the antibodies to EV tumor marker(s) and the antibodies to the drug-resistance biomarker(s) can be applied to the sample and/or the EV-enriched sample at the same time. In some embodiments, the antibodies to the EV tumor marker(s) are applied to the sample and/or EV-enriched sample prior to the antibodies to the drug-resistance biomarker(s) are applied to the sample and/or EV-enriched sample. In some embodiments, the antibodies to the EV tumor marker(s) are applied to the sample and/or EV-enriched sample after to the antibodies to the drug-resistance biomarker(s) are applied to the sample and/or EV-enriched sample.

The sample from the subject can be enriched for EVs using capture antibodies of selected EV tumor markers, e.g., as described herein, wherein the capture antibodies of the selected EV tumor markers are embedded in or attached to a substrate. In some embodiments, to enrich a sample for EVs (e.g., tEVs), the sample is applied to the substrate containing the EV tumor marker capture antibodies to create an EV sample enriched in tumor-derived EVs having the selected tumor markers. After tEVs are captured by the capture antibodies, drug-resistance biomarkers can be detected, e.g., using antibodies can be applied to the tEV-enriched sample in order to determine a level or relative level of drug-resistance biomarker(s)-positive tEVs. In some embodiments, EV enrichment described herein can be combined to include other methods for EV enrichment known in the art.

In some embodiments, enriching EVs (e.g., tEVs) and probing the tEVs for levels of drug-resistance biomarker(s) can be carried out at multiple time points (e.g., over time or longitudinally). For example, enriching EVs (e.g., tEVs) and probing the tEVs for levels of drug-resistance biomarkers can be carried out at one, two, three, four, five, or more time points. In some embodiments, the level (as determined by antibody detection) of drug-resistance biomarker(s) at a first time point can be used to determine the relative level of drug-resistance biomarker(s) at a second time point by comparing the amount of drug-resistance biomarker(s) signal at the second time point to the drug-resistance biomarker(s) signal of the first time point and noting an increase or decrease of drug-resistance biomarker(s) signal. Similarly, the amount of drug-resistance biomarker(s) signal at a third (or fourth, or fifth, etc.) time point can be compared to the amount of drug-resistance biomarker(s) signal at the first time point, or the drug-resistance biomarker(s) signal at the third (or fourth, or fifth, etc.) time point can be compared to the amount of drug-resistance biomarker(s) signal at any previous time point to analyze whether there are any trends in drug-resistance biomarker(s) signal over time.

In some embodiments, a relative increase in levels of drug-resistance biomarker(s)-positive (e.g., P-gp and/or survivin-positive) tEVs over a previous level of drug-resistance biomarker(s)-positive (e.g., P-gp and/or survivin-positive) tEVs indicates cancer cells in the subject are in the process of becoming, or have become, resistant to chemotherapeutic drugs, for example the chemotherapy used to treat the subject's cancer. In some embodiments, a relative decrease or no significant change in levels of drug-resistance biomarker(s)-positive (e.g., P-gp and/or survivin-positive) tEVs over the previous level of drug-resistance biomarker(s)-positive (e.g., P-gp and/or survivin-positive) tEVs indicates cancer cells in the subject have not become resistant to chemotherapeutic drugs (e.g., the chemotherapy used to treat the subject's cancer).

The relative levels of drug-resistance biomarker(s) and biomarker(s)-positive tEVs thus can be used to determine whether the subject receives additional chemotherapeutic treatments comprising the same chemotherapeutic agent used to treat the subject's cancer between the previously evaluated timepoints or receives a different treatment using a different chemotherapeutic agent (or other treatment modality, such as immunotherapy, radiotherapy, or surgical resection).

In some embodiments, a relative increase in drug-resistance biomarker(s) or biomarker(s)-positive (e.g., P-gp and/or survivin-positive) tEVs over a previous level of drug-resistance biomarker(s)-positive (e.g., P-gp and/or survivin-positive) tEVs indicates whether the subject should continue to receive treatment with the same chemotherapy drug. In some embodiments, a relative increase in drug-resistance biomarker(s)-positive tEVs over the previous level of drug-resistance biomarker(s)-positive tEVs indicates the subject should not receive treatment with the same chemotherapy drug (e.g., further treatment with the same chemotherapy drug). In some embodiments, a relative increase in drug-resistance biomarker(s)-positive EVs over the previous level of drug-resistance biomarker(s)-positive tEVs indicates the subject should not receive treatment with the same chemotherapy drug as previously used for the subject (e.g., the subject should be treated with a chemotherapy drug not being used, or not previously used for that subject). In some embodiments, the subject only receives further chemotherapy treatments with the same drug if there is a decrease or no change in drug-resistance biomarker(s)-positive tEVs over the previous (or any previous) level of drug-resistance biomarker(s)-positive tEVs. In some embodiments, the subject only receives further chemotherapy treatments with the same drug if there is not an increase in drug-resistance biomarker(s)-positive tEVs over the previous (or any previous) level of drug-resistance biomarker(s)-positive tEVs. In these conditions, administration of further treatments with the same chemotherapy drug is dependent on the relative level of drug-resistance biomarker(s)-positive tEVs over the previous (or any previous) level of drug-resistance biomarker(s)-positive tEVs.

Types of chemotherapeutic agents that may be given to the subject include, but are not limited to microtubule targeting agents such as taxanes including paclitaxel and docetaxel, maytansine, and eribulin, and vinca alkaloid agents (e.g., vindesine, vinblastine, vinorelbine, vincristine); nitrosoureas (e.g., fotemustine, carmustine (BCNU), or lomustine (CCNU)); anthracyclines (e.g., doxorubicin, aldoxorubicin, epirubicin, and pegy lated liposomal doxorubicin); alkylating agents (e.g., melphalan, dacarbazine (DTIC), temozolomide (TMZ), and oxazaphosphorines such as ifosfamide, cyclophosphamide, trofosfamide, evofosfamide, and palifosfamide); trabectedin; platinum-containing agents such as cisplatin or carboplatin; cytotoxic nucleoside analogs such as gemcitabine; methotrexate; etoposide; pemetrexed, as well as other small molecules chemotherapeutics such as crizotinib, ceritinib, alectinib, brigatinib, loratinib, capmatinib, tepotinib, gefitnib, erlontinib, lapatinib, icotinib, afatinib, osimertinib, neratinib, dacomitinib, almonertinib, tucatinib, medostaruin, gliteritinib quizartinib, pexidartinib, sorafenib, sunitinib, pazopanib, vandetanib, avitinib, cabozantinib, regorafenib, apatinib, Lenvatinib, tivozanib, fruquintinib, nintedanib, anlotinib, erdafinib, pemigatinib, avaprinib, ripretinib, selpercatinib, pralsetnib, larotrectinib, entrectnib, imatinib, dasatinib, nilotinib, bosutinib, radotinib, ponatinib, ibrutinib, acalabrutinib, zanubrutinib, ruxolitinib, fedratinib, vemurafenib, dabrafenib, encorafenib, trametinib, cobimetinib, binimetinib, selumetinib, palbociclib, ribociclib, abemaciclib, idelalisib, copanlisib, duvelisib, alpelisib, temsirolimus, evrolimus, sirolimus, tazemetostat, vorinostat, romidepsin, belinostat, tucidinostat, panobinostat, enasidenib, ivosidenib, venetoclax, vismodegib, sonidegib, glasdegib, bortezomib, carfilzomib, and ixazomib, see, e.g., Zhong et al., Signal Transduct Target Ther. 2021; 6: 201; Tian and Yao, Front Pharmacol. 2023; 14: 1199292. In some embodiments, the drug-resistance biomarker(s) detect a subject's drug resistance of one or more of the chemotherapeutic agents listed above. In some embodiments, a subject may be or may have taken one of the chemotherapeutic agents described above prior to, during, or after monitoring drug resistance using tEVs and drug-resistance biomarker(s) as described herein. In some embodiments, monitoring drug resistance in subject includes monitoring drug resistance to one of the chemotherapeutic agents described above.

As used herein, the terms “cancer,” “tumor” or “tumor tissue” has the meaning as understood by one skilled in the art. A cancer, tumor, or tumor tissue can include tumor cells that are neoplastic cells with abnormal growth properties. Tumors, tumor tissue, and tumor cells can be benign or malignant. Cancer can include primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Examples of cancer include, but are not limited to, carcinoma, lymphoma, blastoma, sarcoma, and leukemia or lymphoid malignancies. Additional examples of such cancers are noted below and include: squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, cholangiocarcinoma, bladder cancer, hepatoma, breast cancer, colon cancer, rectal cancer, colorectal cancer, endometrial cancer or uterine carcinoma, salivary gland carcinoma, kidney or renal cancer, prostate cancer, vulvar cancer, thyroid cancer, hepatic carcinoma, anal carcinoma, penile carcinoma, as well as head and neck cancer.

One of the benefits of the longitudinal monitoring of drug-resistance of the currently described methods is that drug-resistance in a subject can be detected prior to an observable increase in size of the subject's cancer (e.g., tumor). With an early detection of drug-resistance, the longitudinal monitoring of drug-resistance of the currently described methods can terminate the toxic treatment early to reduce side effects or minimize unnecessary treatment. The efficacy of the longitudinal monitoring of drug-resistance of the currently described methods for predicting drug-resistance can be at least 95%. Other benefits of the longitudinal monitoring of drug-resistance of the currently described methods include predicting drug treatment efficacy, minimizing the detection of residual diseases, and facilitating early detection of disease recurrence.

### Extracellular Vesicles (EVs)

Extracellular vesicles (EVs) are lipid-based microparticles, nanoparticle, or protein-rich aggregates present in a sample (e.g., a biological fluid) obtained from a subject. Extracellular vesicles are also referred to in the art and herein as exosomes, microvesicles, or nanovesicles. Extracellular vesicles can include membrane vesicles secreted from cell surfaces (ectosomes), internal stores (exosomes), cancer cells (oncosomes), or released as a result of apoptosis and cell death. In addition to lipid membranes, depending on their cell or tissue of origin, EVs can include additional components such as lipoproteins, proteins, nucleic acids, phospholipids, amphipathic lipids, gangliosides and other particles contained within the lipid membrane or encapsulated by the EVs.

All cells likely release, secrete, or shed EVs, making them useful clinical diagnostic and therapeutic targets for a range of diseases. Non-limiting examples of normal or cancer cell types that can release EVs include liver cells (e.g., hepatocytes), lung cells, spleen cells, pancreas cells, colon cells, skin cells, bladder cells, eye cells, brain cells, esophagus cells, cells of the head, cells of the neck, cells of the ovary, cells of the testes, prostate cells, placenta cells, epithelial cells, endothelial cells, adipocyte cells, kidney cells, heart cells, muscle cells, blood cells (e.g., white blood cells, platelets), and combinations of the foregoing. Because EVs are involved in cell-cell communication, their characterization casts light upon their role in normal physiology and pathology. EVs in biological fluids including saliva, urine, plasma, and serum can be interrogated as biomarkers of any number of cancers described herein. An EV enriched or isolated for having particular tumor markers can be referred to as a tumor-derived EV (tEVs).

In some embodiments, an extracellular vesicle is between about 20 nm to about 200 nm in diameter. Individual EVs have ˜1/10,000 the surface area and ˜1/1,000,000 the volume of a whole cell and are therefore difficult to detect using single cell analysis tools, including conventional flow cytometry. As a result, most proteomic and genomic analysis is performed in bulk on thousands or millions of EVs. However, EVs in biofluids come from many different cell types, and from different locations from within the cell (exosomes secreted from intracellular multi-vesicular bodies, ectosomes/microvesicles shed from the plasma membrane surface, membrane fragments released as a result of cell apoptosis, necrosis, etc.). Thus, in a bulk analysis, the signature from tumor EVs may be lost in the background of vesicles from other sources, and methods of enriching tEVs help capture a more robust tEV picture.

EVs represent new opportunities as circulating cancer biomarkers. These cell-derived membrane-bound vesicles contain protein and nucleic acid cargo, providing a representative ‘snapshot of the content of the secreting cells. Large abundance and ubiquitous presence of tumor-derived EVs (tEVs) in bodily fluids (e.g., blood, urine) have shown the potential use of EVs as readily accessible biomarkers. Namely, tumor-derived EV (tEV) analyses can be minimally invasive for repeated sampling and afford relatively unbiased readouts of the entire tumor, less affected by the scarcity of the samples or intratumoral heterogeneity. This suggests that tEVs may have particular utility for longitudinal disease monitoring and early detection of relapse. Previous studies showed that both the amount and molecular profiles of tEVs were shown to correlate with tumor burden as well as treatment efficacy. Therefore, EVs can function as a novel biomarker for liquid biopsy in personalized medicine. However, EVs are relatively new targets for analytical assays in clinics and possess unique physical and biological traits. They fall in size range much smaller than cells but larger than proteins and exist in a highly heterogeneous biological background. These properties impose technical difficulties, which often lead to variable findings. Furthermore, identifying cell-specific (e.g., tumor origins) EVs and interrogating drug-resistant markers within the subpopulation require multiplexed analysis, ideally in a single EV resolution.

Molecular characterization of single EVs is technically challenging. Most EVs are small vesicles (<200 nm) with limited numbers of epitopes and surface areas for labeling (i.e., weak detectable signals), which often requires sophisticated multi-step signal amplification strategies, such as DNA barcodes or enzymatic signal amplification (digital ELISA). Flow cytometry often underestimates EV counts because many small vesicles (<200 nm) could be missed due to their weak light scattering, or a swarm of vesicles could be counted as a single event. More importantly, many of these methods are suboptimal for detecting and quantifying very rare tEVs in excessive background EVs and particles. Multiplexed analysis of EVs is especially critical to identify the origin of individual EVs (e.g., tEVs) and molecularly profile biomarkers associated with targeted therapies and mutations.

Thus, provided herein are methods of isolating and enriching tumor EV particles, in order to monitor and/or evaluate whether tumor cells in a subject have become resistant to chemotherapeutic drugs over time.

### EV Tumor Markers

Extracellular vesicles of tumor origin can carry tumor markers. An EV tumor marker profile can indicate the origin of a cancer or the type of cancer cells found in a subject. For example, MUC1, HER2, EGFR, and EpCAM are four markers that can be used to identify breast cancer cells in a subject. Many EVs secreted by these breast cancer cells also contain these four tumor markers. Therefore, monitoring EVs that comprise one or more of MUC1, HER2, EGFR, and EpCAM, e.g., tumor EVs (tEVs), in a subject can give information regarding a subject's breast cancer, including development of drug resistance. Thus herein, we provide methods wherein monitoring tEVs comprising one or more of MUC1, HER2, EGFR, and EpCAM in a subject can be used to determine whether a subject's cancer is becoming or has become resistant to chemotherapy. In some embodiments, monitoring tEVs expressing all four MUC1, HER2, EGFR, and EpCAM markers can be used to determine whether a subject's cancer is becoming or has become resistant to chemotherapy.

Other EV tumor markers include: EpCAM, miRNA-21, and CD24 (e.g., as tumor markers of ovarian cancer); EpCAM, EGFR, MUC1, WNT2, and GPC1 (e.g., as tumor markers of pancreatic ductal adenocarcinoma (PDAC)); EGFR and EGFRvIII (e.g., as tumor markers of glioblastoma (GBM)); and EpCAM, EGFR, and MUC1 (e.g., as tumor markers of cholangiocarcinoma); see, e.g., Im et al., Label-free detection and molecular profiling of exosomes with a nano-plasmonic sensor, Nat. Biotech. 2014; Yang, et al, Multiparametric plasma EV profiling facilitates diagnosis of pancreatic malignancy. Sci. Transl. Med. 9, eaal3226 (2017); Min et al., Plasmon-enhanced biosensing for multiplexed profiling of extracellular vesicles, Adv. Bio., 2000003 (2020); Jeong, et al., Plasmon-enhanced single extracellular vesicle analysis for cholangiocarcinoma diagnosis, Adv. Sci., 2205148 (2023). Hong, et al., CRISPR/Cas13a-Based MicroRNA Detection in Tumor-Derived Extracellular Vesicles, Adv. Sci., 2023. Any of these tumor markers can be used as EV tumor markers in the methods described herein. Other EV tumor markers including miRNA and other non-coding RNAs can be found in the art and readily appreciated by the skilled artisan. See, e.g., Huang, et al., Non-coding RNA derived from extracellular vesicles in cancer immune escape: Biological functions and potential clinical applications, Cancer Lett., 2021.

### Drug-Resistance Biomarkers

Some cancer cells become drug resistant over time. If the cancer cells in a subject become drug resistant to the chemotherapy drug used to treat the subject's cancer, then the cancer may progress and the subject can experience a worsening of the cancer and/or cancer symptoms. Therefore, it is important to understand whether a subject is in the process of becoming resistant to any chemotherapeutic drugs the subject is taking.

There are drug-resistant biomarkers, detection of which indicate that the cells in which the sample was taken have become resistant to one or more drugs used to treat cancer. One example of a drug-resistant biomarker is P-glycoprotein (P-gp). As shown herein, increased transcription and/or increased translation of P-gp has been observed in drug-resistant cancers. Therefore, because overexpression of P-gp is common in many drug-resistant cancers, P-gp is a common drug-resistant biomarker. Similarly, survivin is a member of the inhibitor of apoptosis (IAP) family. Overexpression of survivin is common in most tumor cell types, but is typically not present in normal, non-malignant adult cells. Because overexpression of survivin contributes to their resistance to apoptotic stimuli, survivin is common drug-resistant biomarker for many drug-resistant cancers.

Provided herein are methods that include using P-gp and/or survivin as drug-resistant biomarkers. The methods described herein include using P-gp and survivin as drug-resistant biomarkers. The methods can include detecting and optionally quantifying the drug-resistance biomarkers using antibodies or antigen binding portions thereof that bind to the drug-resistance biomarkers. In some embodiments, the antibodies or antigen binding portions thereof that bind to the drug-resistance biomarkers are detectable labeled, e.g., fluorescently labeled. In some embodiments the antibodies or antigen binding portions thereof that bind to the drug-resistance biomarkers are labeled with a secondary antibody.

### EXAMPLES

The materials and methods described here have been used to generate the examples described herein.

### Materials and Methods

The following materials and methods were used in the Examples below.

### Drugs

Paclitaxel (Selleckchem, USA) was dissolved in dimethyl sulfoxide (DMSO; AppliChem, Barcelona, Spain) and stored at −80° C., according to the manufacturer's instructions. Immediately prior to use, an aliquot was diluted at required concentrations.

### Cell Culture

The human breast cancer cell lines, including HCC1954, BT474, MCF7, MDA-MB-231, HCC1937 cells, and normal breast cell, Hs371T, were purchased from American Type Culture Collection (ATCC) and cultured at 37° C. in 5% CO2. HCC1954, BT474, and HCC1937 cells were grown in RPMI-1640 (Hyclone), and MCF7, MDA-MB-231, and Hs371T cells were cultured in DMEM (Cyclone). All complete media contained 10% fetal bovine serum (FBS, ThermoFisher Scientific), 100 U/mL penicillin, and 100 μg/mL streptomycin (Millipore Sigma).

Establishment of Paclitaxel-Resistant Subtype from Parental (Control) Cell Line

To develop the paclitaxel resistance subtype, cells were continually exposed to stepwise increases in the concentration of paclitaxel over 16 to 18 weeks. Briefly, HCC1954 cells were seeded at a density of ˜5×10⁵/mL in a T75 cell culture flask with 10 mL complete growth medium. After 4-6 hours of incubation, relatively low concentrations of paclitaxel (ranging from 1 to 150 nM) were added into the medium. Cells were left in paclitaxel for three days or until a stable cell re-population formed. Regular medium replenishment was performed throughout this period. The paclitaxel concentration was then increased by 0.5 to 2-fold. This stepwise dose escalation continued for 16 to 18 weeks until the paclitaxel concentration reached at least ten times the starting concentration. After that, the HCC1954 paclitaxel-resistant cell line (HCC1954 REPX) was maintained in the same medium as their parental cell line.

### Drug Sensitivity

Drug sensitivity was determined by the 3-(4, 5-dimethylthiazol-2-yl)-2, 5-diphenyl tetrazolium bromide (MTT) colorimetric assay. First, cells were counted with a hemocytometer, seeded in a 96-well plate, and cultured for 24 h. After that, cells were treated with paclitaxel for 72 h: paclitaxel (1.00E-06-10 UM). Subsequently, MTT dye (Sigma-Aldrich; Merck KGaA) at a final concentration of 0.5 mg/ml was added for 3 hours. This was followed by replacing the medium and dissolving the formazan crystals with DMSO. The optical density (OD; absorbance at 540 nm) was measured using a plate reader (Tecan). Data (mean±SD of at least three independent experiments performed in triplets) are presented as the relative proliferation as a function of time after seeding.

### EV Isolation and Characterization

EVs were isolated as previously reported [Min et al., 2020]. Briefly, the cells were grown unto 80-90% confluence in a complete medium and washed twice with PBS to remove floating cells and the vesicles from FBS. The cells were then incubated in a conditioned medium supplemented with 1% Exosome-depleted FBS, 100 U/mL penicillin, and 100 μg/mL streptomycin for 48 h. The conditioned medium underwent filtration to remove cells and apoptotic bodies and then concentrated using centrifugal filter units (Centricon Plus-70 Centrifugal Filter (MWCO=10 kDa, Millipore Sigma)). Next, the concentrates were processed using size exclusion chromatography (SEC), as previously reported [Jeong et al., 2023].

Similar procedures were applied to plasma samples, initially involving centrifugation to eliminate cell debris. EV isolation from plasma samples employed a modified SEC column, known as dual-mode chromatography (DMC). Plasma samples were centrifuged at 2,000×g for 3 min to remove cell debris, and supernatants were collected. As previously reported, five hundred microliters of supernatants diluted in 500 μL of PBS were used for isolation experiments by isolation size exclusion chromatography (EDMC) [Woo et al., 2022].

Finally, the EVs fractions were concentrated with the Amicon Ultra-2 Centrifugal Filter (MWCO=10 kDa, Millipore Sigma) at 3,500×g for 30 min at 4° C. and stored until use at −80° C.

NanoSight LM10 (Malvern) equipped with a 405 nm laser was used. Samples were diluted in fPBS to obtain the recommended particle concentration (25-100 particles/frame). For each test sample, three 30-sec videos were recorded (camera level, 14). Recorded videos were analyzed by NTA software (version 3.2) at a detection threshold of 3.

### Quantitative Real-Time PCR

Total RNA was extracted with TRIzol™ LS Reagent (Thermo Fisher Scientific), according to the manufacturer's instructions. The RNA concentration was determined with NanoDrop Spectrophotometer (NanoDrop Technologies) and the dissolved RNA was stored at −20° ° C. before use. cDNA synthesis was performed with iSuperScript™ III First-Strand Synthesis SuperMix Kit (Thermo Fisher Scientific), following supplier's instructions. For real-time PCR, cDNA was amplified using iSYBR™ Green PCR Master Mix Kit (Thermo Fisher Scientific).

The quantitative PCR reactions were performed in triplicate for each sample-primer set, and the mean of the three experiments was used as the relative quantification value. To accurately determine the starting copy number regardless of the precise amount and qualities of input DNA, we also quantified internal control genes (GAPDH) in each single reaction. Primers were as follows:
TUBA1A/α-tubulin forward: 5′-CGGGCAGTGTTTGTAGACTTGG-3′ (SEQ ID No: 1) and reverse: 5′-CTCCTTGCCAATGGTGTAGTGC-3′ (SEQ ID No: 2):
TUBB3/βIII-tubulin forward: 5′-GCGAGATGTACGAAGACGAC-3′ (SEQ ID No: 3) and reverse: 5′-TTTAGACACTGCTGGCTTCG-3′ (SEQ ID No: 4);
CIC-3 forward: 5′-CCTCTTTCCAAAGTATAGCAC-3′ (SEQ ID No: 5) and reverse: 5′-TTACTGGCATTCATGTCATTTC-3′ (SEQ ID No: 6);
ABCB1/P-gp forward: 5′-TGCTCAGACAGGATGTGAGTTG-3′ (SEQ ID No: 7) and reverse: 5′-AATTACAGCAAGCCTGGAACC-3′ (SEQ ID No: 8):
ABCG2/BCRP forward: 5′-TATAGCTCAGATCATTGTCACAGTC-3′ (SEQ ID No: 9) and reverse: 5′-GTTGGTCGTCAGGAAGAAGAG-3′ (SEQ ID No: 10):
Survivin forward: 5′-ACCGCATCTCTACATTCAAG-3′ (SEQ ID No: 11) and reverse: 5′-CAAGTCTGGCTCGTTCTC-3′ (SEQ ID No: 12):
PTGES3 forward: 5′-CAAATGATTCCAAGCATAAAAGAAC-3′ (SEQ ID No: 13) and reverse: 5′-GGTAAATCTACATCCTCATCACCAC-3′ (SEQ ID No: 14).
CCND1/cyclin D1 forward: 5′-GGATGCTGGAGGTCTGCGA-3′ (SEQ ID No: 15) and reverse: 5′-AGAGGCCACGAACATGCAAG-3′ (SEQ ID No: 16); and
GAPDH: forward: 5′-ACAGTCCAGCCGCATCTTC-3′ (SEQ ID No: 17) and reverse: 5′-GCCCAATACGACCAAATCC-3′ (SEQ ID No: 18).

To isolate small RNA from cells and their derived EVs mirVana miRNA Isolation Kit (Thermo Fisher Scientific) and miRNeasy Serum/Plasma Kit (Qiagen) were used according to each manufacturer's isolation procedure. miRNA concentration was quantified by Qubit® microRNA Assay Kit (Thermo Fisher Scientific). RT-qPCR was performed using TaqMan MicroRNA primers and kits (Thermo Fisher Scientific) and the CFX Opus Dx Real-Time CR Detection Systems (Bio-Rad). The manufacturer has extensively tested and established specificities of U6 small RNA and miRNA primers. The Catalog Numbers of reagents are as follows: hsa-miR-9/ID000583: #4427975: hsa-miR-421/ID002700: #4427975: hsa-miR-21/ID000397: #4427975; and U6snRNA/ID001973: #7840156. Briefly, each reverse transcription reaction used 10 ng of input RNA, and the reaction was set up according to the manufacturer's specifications. A master mix was prepared with 0.15 μl of 100 mM deoxynucleoside triphosphate (dNTP), 1 μl of MultiScribe reverse transcriptase (50 U/μl), 1.5 μl of 10×reverse transcription buffer, 0.19 μl of RNase inhibitor (20 U/μl) and 4.1 μl of nuclease-free water per reaction. Then, 7 μl of master mix was combined with 3 μl of 5×reverse transcription TaqMan assay primer and 5 μl of RNA (10 ng total input). Thermal cycling conditions for reverse transcription were as follows: 16° ° C. for 30 min, 42ºC for 30 min, 85ºC for 5 min and 4° C. hold. Subsequent qPCR was performed using 1.33 μl of cDNA and the TaqMan Fast Advanced Master Mix according to the manufacturer's specifications. In brief, each qPCR reaction consisted of 10 μl of 2× TaqMan Fast Advanced Master Mix, 1 μl of 20× qPCR TaqMan assay primer, 7.67 μl of nuclease-free water and 1.33 μl of cDNA. Thermal cycling for qPCR was performed as follows: 50° C. for 2 min, 95° C. for 20 s, and 40 cycles of 95ºC for 1 s and 60° ° C. for 20 s.

Relative gene expression levels between experimental groups were determined using the comparative Ct (2-ΔΔCt) method after normalizing to reference genes [Livak and Schmittgen, 2001].

### EV Capture on Beads

50 μl of each fraction isolated by SEC (diluted or not) or by ultracentrifugation were incubated with 0.25 μl of aldehyde/sulfate-latex beads (ø=4 μm: 5.5×106 particles/ml; Invitrogen) for 15 min at RT. Dilutions of the EV samples were performed with the same buffer used for their elution (PBS). Bead concentration was chosen to be large enough to be easily detected by the flow cytometer having enough events for proper statistics (over 5000), and small enough to use a small volume of sample.

### Flow Cytometry Staining and Analysis

EVs and cells were stained using the same antibodies and procedure. Cells were prepared for flow staining by fixing and permeabilizing 500,000 cells per antibody condition in Fix and Permeabilization 4% paraformaldehyde and 1× Perm/Wash (ThermoFisher) in PBS for 15 min at room temp on a nutating mixer. Cells were washed in PBS/1% BSA by centrifugation at 400×g for 3 min (cells) or 1000× g for 1 min (EVs on beads). Samples were resuspended in 100 μl PBS/1% BSA or in primary antibody diluted in the same buffer/volume and incubated for 30 min on a plate shaker set to medium speed (see Table 1 for a list of antibodies). Cells or EVs were then resuspended in 100 μl of the appropriate secondary antibody diluted 1:1000 in PBS/1% BSA and incubated for 30 min (protected from light) on a plate shaker. Samples were again pelleted, washed twice with 150 μl PBS/1% BSA, and resuspended in 200 μl PBS/1% BSA for flow analysis. Samples were measured using a BD LSR II flow cytometer (BD Biosciences). Pre-gating was done to ensure single cells or beads were analyzed, and a total of 10,000 events were collected within the gated area. FlowJo (v10) was used to analyze samples by measuring the median fluorescence intensity for proteins of interest and corresponding isotype controls. For comparisons, fold change in median fluorescence intensity was calculated by dividing the signal for a protein of interest over the isotype control.

TABLE 1
Flow cytometry antibodies

| | | | |
|---|---|---|---|
| Name | Isotype | Cat n. | Source |

List of the employed primary antibodies

| | | | |
|---|---|---|---|
| Anti-P-gp | Rabbit | ab235954 | Abcam |
| Anti-Survivin | Mouse IgG1 | TA501245 | ThermoFisher Scientific |
| Anti-Cyclin D1 | Mouse IgG2a | 681902 | BioLegend |
| Anti-TSG101 | 67381-1-Ig | 67381-1-Ig | Proteintech Group |

List of the employed Secondary Antibodies

| Reagent | Cat n. | Source |
|---|---|---|
| Anti-rabbit IgG (H + L), F(ab′)₂ | 4412S | Cell Signaling |
| Fragment (Alexa Fluor ® 488) | | Technology |
| Goat anti-Mouse IgG (H + L) | A21236 | ThermoFisher Scientific |
| (Alexa Fluor ™ 647) | | |

### Fluorescent Labeling of EVs

EVs were labeled with AF555 dye as previously reported with minor modifications [Ferguson et al., 2022; Spitzberg et al., 2023]. Briefly, 3 μL of 300 ng of EVs in PBS were mixed with 2 μL of 100 mM sodium bicarbonate (Millipore Sigma) and 0.2 μL of TFP-AF555 dye [mixture of 0.22 UM of Azido-dPEG®12-TFP ester (Quanta Biodesign) and 0.2 μM of AFDye 555 DBCO (Click Chemistry Tools) in a equal volume] at RT for 1 hr in a dark condition. The labeled EVs were diluted with PBS in an appropriate concentration before loading on the substrates.

### Functionalization and Immunofluorescence Staining

3D plasmonic nanostructure composed of spherical AU nanoparticles on 3D Au nanopillars (NPOP) substrates were fabricated. See Park, et al. Self-assembly of nanoparticle-spiked pillar arrays for plasmonic biosensing, Adv. Funct. Mater., 1904257 (2019). For the analysis of cell line-derived EVs, all EVs were attached to the surface. The NPOP surface was functionalized using MUA, SH-PEG-COOH (0.4 kDa), and SH-PEG-COOH (1.0 kDa), respectively. In brief, 10 mM of 11-Mercaptoundecanoic acid (Millipore Sigma) and 1-Octanethiol (Millipore Sigma) were mixed in absolute ethanol and applied onto the NPOP substrate for 2 hours, followed by sequential washing with absolute ethanol and water. Additionally, for SH-PEG-COOH (0.4 and 1.0 kDa) treatment, 0.25 mM of each SH-PEG-COOH (Nanocs) prepared in water was applied onto the NPOP substrate for 4 hours and washed with water. Subsequently, the NPOP substrates were treated with a mixture of 50 mM 1-ethyl-3-(3-dimethylaminopropyl)carbodiimide hydrochloride (EDC, ThermoFisher Scientific) and 125 mM sulfo-N-hydroxysulfosuccinimide (NHS, ThermoFisher Scientific) in 0.1M MES (pH 6.0) for 7 minutes. After activation, TFP-AF555-labeled EVs were loaded onto the NPOP substrate and incubated for 30 minutes. The EVs were then fixed and permeabilized using 4% paraformaldehyde (Electron Microscopy Sciences) and 1× Perm/Wash buffer (BD bioscience) in PBS for 10 minutes, followed by a 30-minute blocking step with BSA in PBS (ThermoFisher Scientific). Subsequent fluorescent labeling of the EVs was achieved through sequential incubation with primary and secondary antibodies, as specified in Table 2.

For plasma-derived EVs, to selectively capture tEVs, the NPOP substrate was pre-coated with QUAD markers (MUC1, HER2, EGFR, and EpCAM). The chips were incubated in a 100 mM sodium citrate solution at room temperature for 1 hour. Following this, the chips were washed with distilled water and dried with nitrogen gas. Then, capture antibodies (QUAD) were mixed in 1% Goat serum/PSB 1× buffer and added to the slide to incubate at room temperature for 1 hour. After 3-4 washes with PBS, the QUAD antibodies were blocked with 10% Goat serum for 20 minutes at room temperature. Subsequently, TFP-AF555-labeled EVs were loaded onto the NPOP substrate and incubated for 1 hour. The EVs were fixed and permeabilized using 4% paraformaldehyde (Electron Microscopy Sciences) and 1× Perm/Wash buffer (BD bioscience) in PBS for 10 minutes, followed by 3-4 washes with PBS. Finally, the EVs were fluorescently labeled through sequential incubation with primary and secondary antibodies, as indicated in Table 2.

Following immunolabeling, the substrates were washed with PBS, and images were captured using a Zeiss upright automated epifluorescence microscope equipped with a 40× (NA=0.95) objective lens and an sCMOS camera (Hamamatsu).

TABLE 2
NPOP antibodies
List of the employed primary antibodies

| Name | Isotype | Cat no. | Source |
|---|---|---|---|
| Anti-P-gp | Rabbit | ab235954 | Abcam |
| Anti-Survivin | Rabbit | 2808T | CST |
| Anti-Epcam | Mouse IgG1 | Ab85987 | Abcam |
| Anti-Muc1 | Mouse IgG1 | MA1-06503 | ThermoFisher |
| Anti-HER2 | Mouse IgG1 | Ab16901 | Abcam |
| Anti-EGFR | Mouse IgG2 | Ab30 | Abcam |

List of the employed Secondary Antibodies

| Reagent | Cat no. | Source |
|---|---|---|
| Anti-rabbit IgG (H + L), F(ab′)₂ | 4414 | CST |
| Fragment (Alexa Fluor ® 647) | | |

### Image Processing

Images were analyzed using ImageJ and custom-built Jupyter Notebook code. Background intensity was subtracted using the rolling ball method (radius=20), and the ImageJ Comdet plugin was used to detect EV locations from the AF555 channel. 3×3 averaged pixel intensity was obtained from AF647 and DL755 channels. IgG control experiments defined the intensity thresholds for AF647 and DL755 channels, and the threshold value was defined by mean+3×standard deviation.

### Example 1. Growth Inhibitory Effect of Paclitaxel on Various Cancer Cell Lines

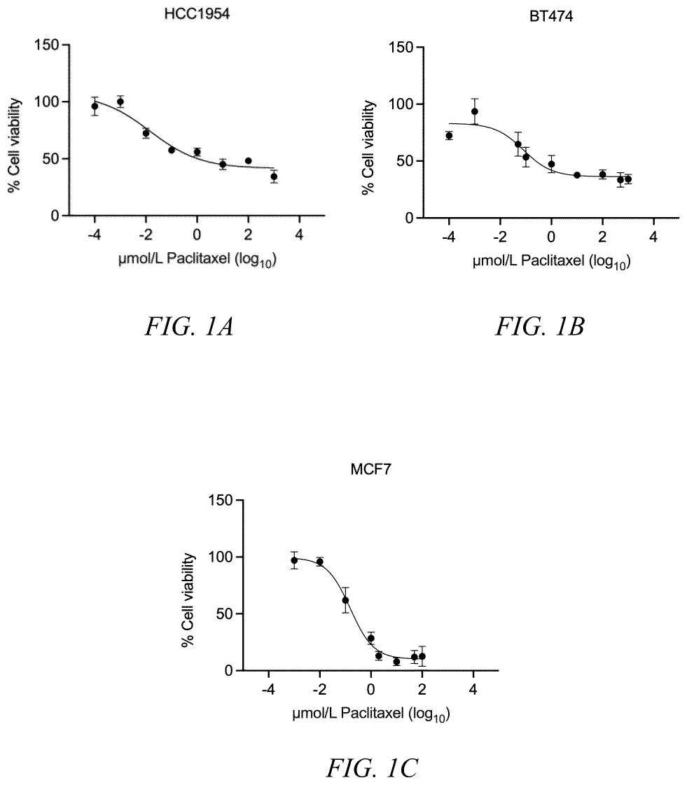

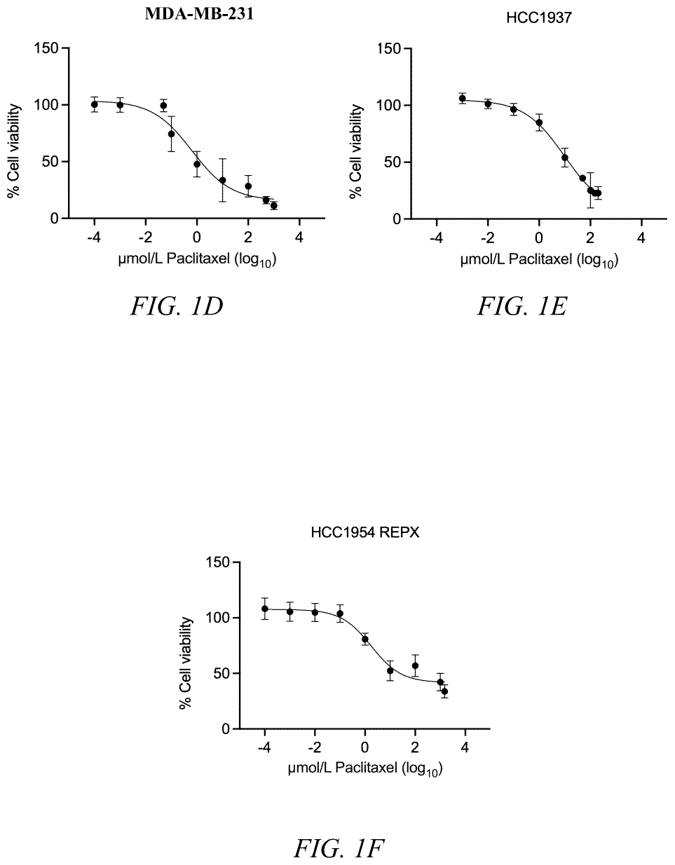

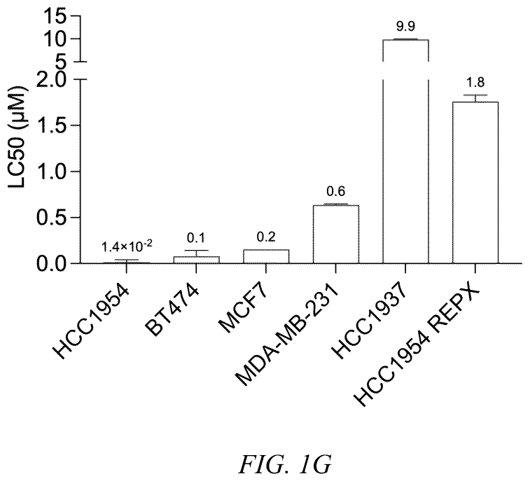

MTT assay was performed for each cell line to determine the paclitaxel inhibitor concentration 50 (IC50) in the six breast cancer cell lines included. The IC50 of paclitaxel in the tested cell modelswas 1.4E-02 μM+0.027 s.d. for HCC1954 (FIG. 1A), 0.1 μM±0.063 s.d. for BT474 (FIG. 1B), 0.2 μM±0.001 s.d. for MCF7 (FIG. 1C), 0.6 μM±0.011 s.d. for MD-MB-231 (FIG. 1D), and 9.9 μM±0.097 s.d. for HCC1937 (FIG. 1E).

To generate a resistance cell model in vitro (HCC1954 REPX), the HCC1954 cell line was treated with increasing concentrations of paclitaxel, and cell viability was re-evaluated after 18 weeks of treatment. A significant increase in paclitaxel IC50 was observed for the HCC1954 REPX cell subtype (1.8 μM±0.072 s.d., FIG. 1F) compared to the parental HCC1954 cell line (1.4E-02 μM±0.027 s.d., FIG. 1G), demonstrating a 128.57-fold increase in paclitaxel-resistance.

### Example 2. Size Characterization of EVs

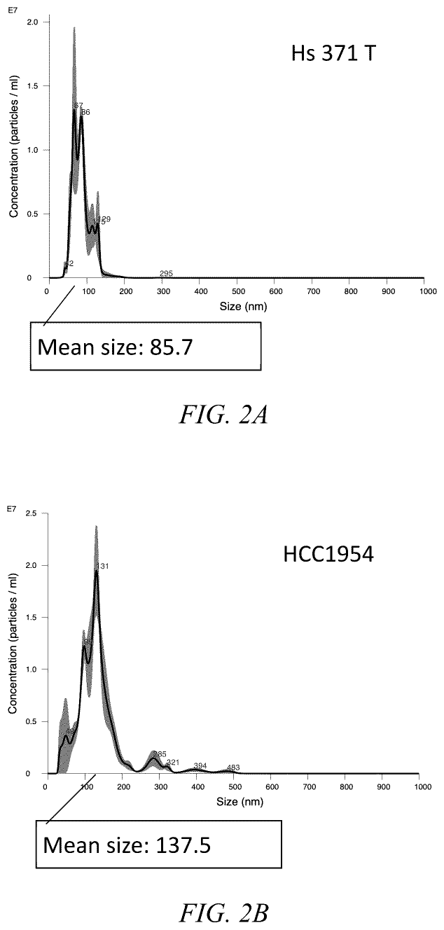

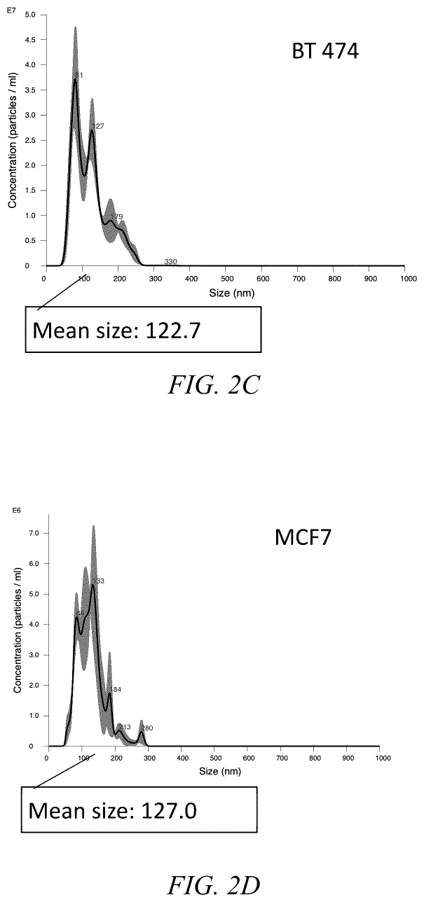

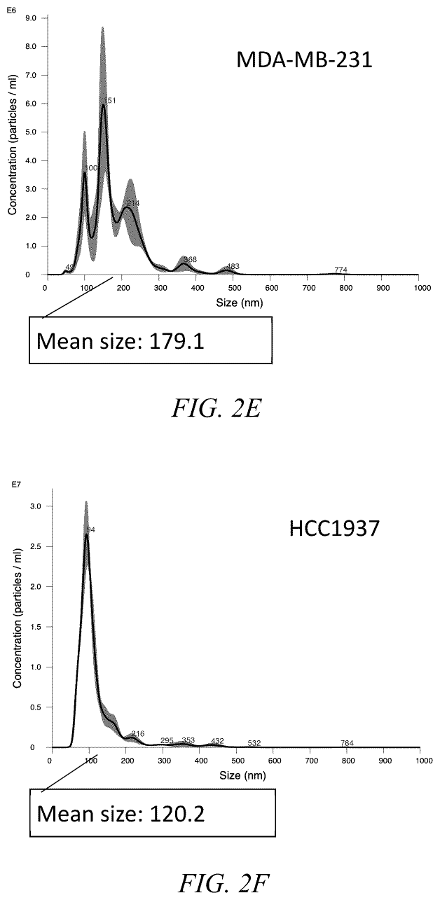

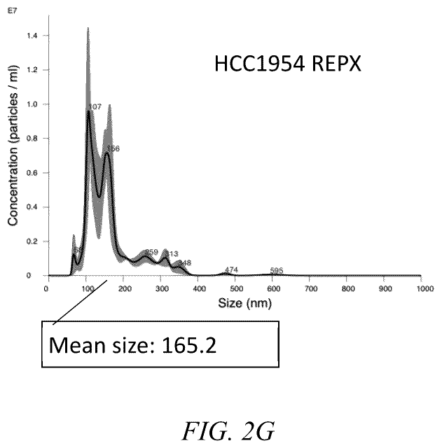

EV fractions were isolated from the seven breast cancer cell lines, HCC1954 (FIG. 2B), BT474 (FIG. 2C), MCF7 (FIG. 2D), MDA-MB-231 (FIG. 2E), HCC1937 (FIG. 2F), and HCC1954 REPX (FIG. 2G), and the normal cell line Hs 371 T (FIG. 2A), and further characterized for the analysis of size distribution and concentration by NTA. The particle population exhibited a high degree of homogeneity in terms of size, falling within the expected range for EVs (range 85.7-179.1 nm).

### Example 3. Comparative Gene Expression Profiles Analyzed Via qPCR

Eleven markers potentially associated with paclitaxel resistance were examined: TUBA1A/α-tubulin, TUBB3/BIII-tubulin, CIC-3, ABCB1/P-gp, ABCG2/BCRP, survivin, PTGES3, CCND1/cyclin D1, miR-9, miR-421, and miR-21. These selected markers underwent evaluation in both cells and their corresponding EVs.

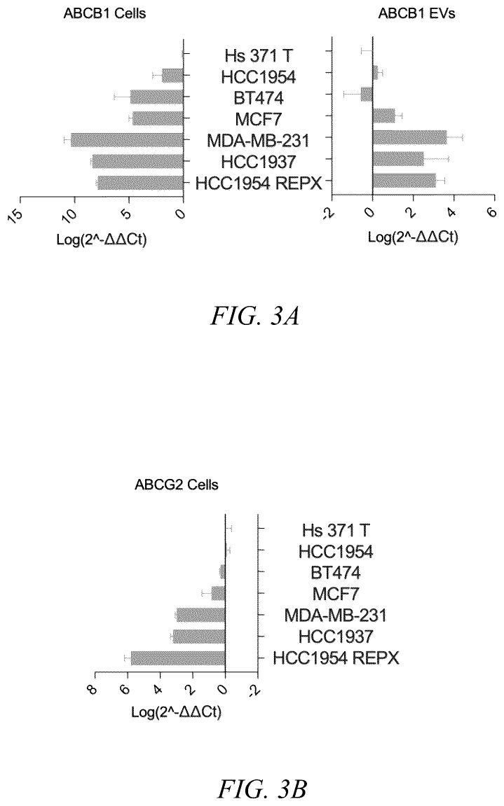

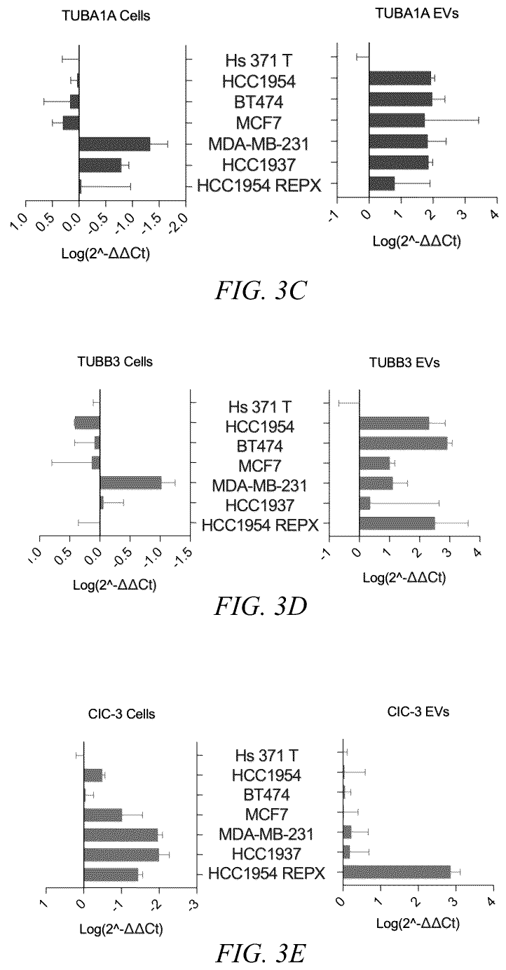

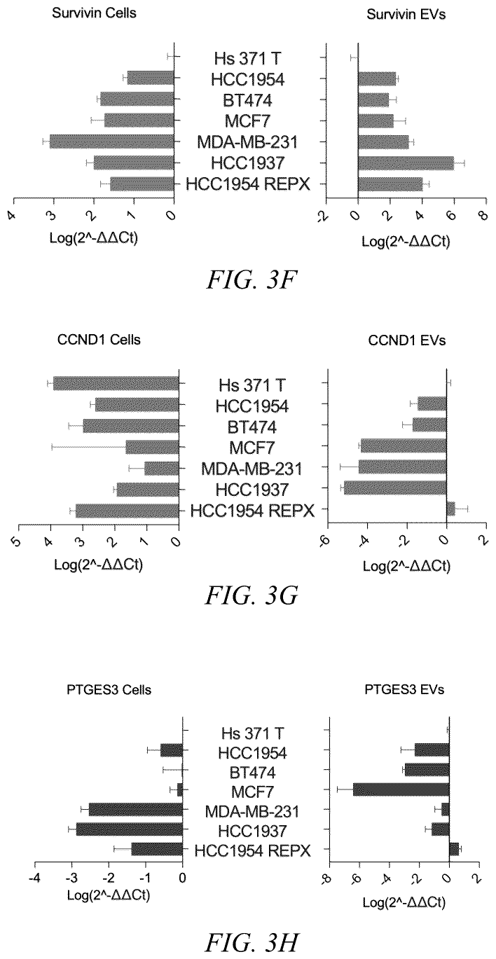

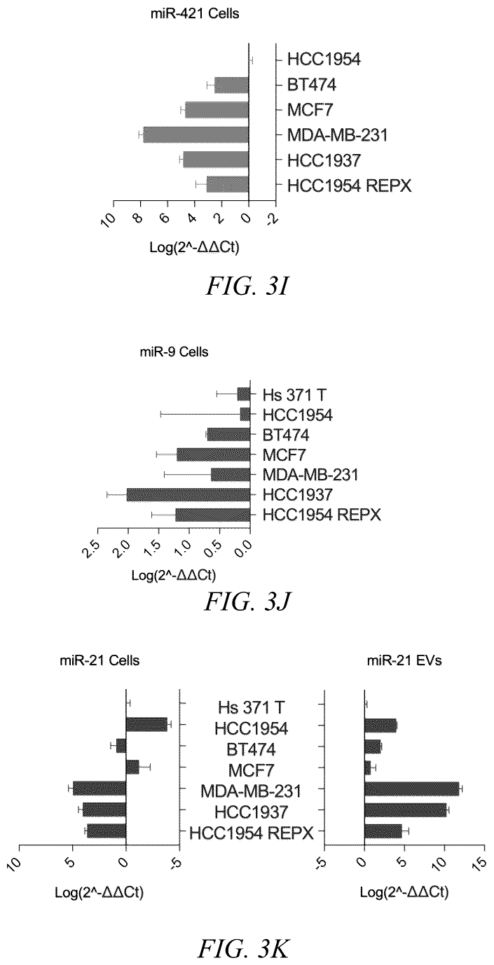

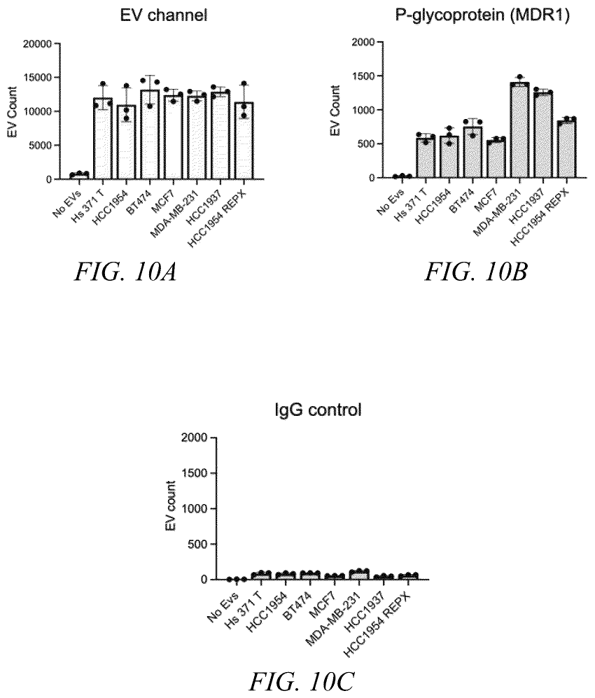

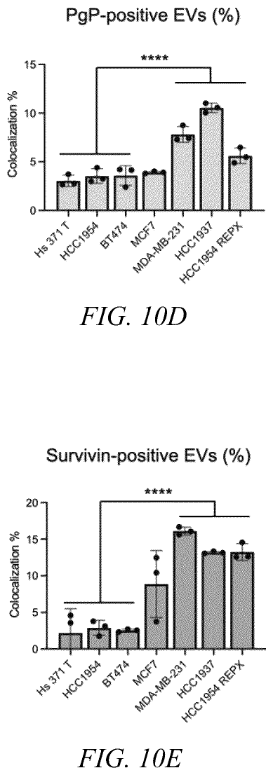

The expression levels of the ABC transporter ABCB1/P-gp (FIG. 3A and FIG. 10B) were notably higher in the less sensitive cell lines, MDA-MB-231, HCC1937, and HCC1954 REX, compared to the more sensitive cell lines, HCC1954, BT474, and MCF7. Furthermore, the expression of ABCB1/P-gp was significantly reduced in the normal cell subtype, Hs 371 T. A similar expression trend was observed in the EVs isolated from the tested cell types (FIG. 3A), demonstrating a direct correlation between increased expression of ABCB1/P-gp and enhanced paclitaxel resistance. In case of ABCG2/BCRP (FIG. 3B), a positive correlation was observed between increased expression of ABCG2/BCRP and higher IC50 value at the cellular level, however, ABCG2/BCRP was not detectable in EVs. These findings affirm extensive research highlighting the interaction of ABCB1/P-gp with paclitaxel resistance and underscore the potential of ABCB1/P-gp in predicting response to paclitaxel.

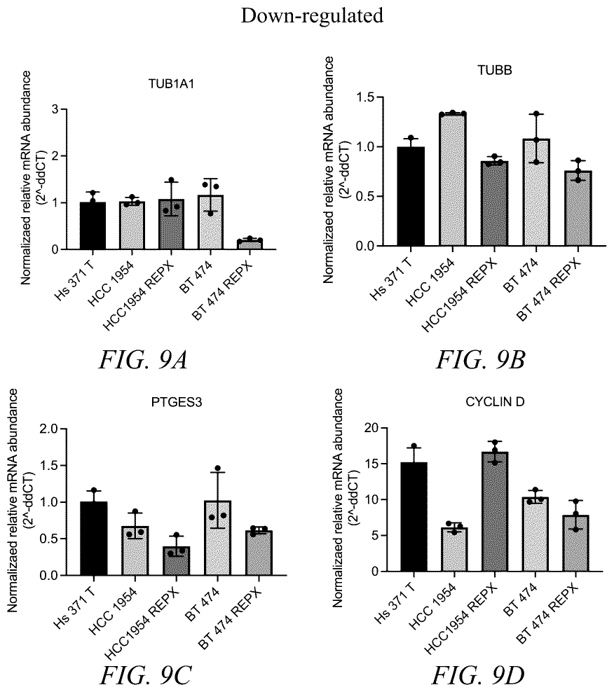

The present results showed a reduced expression of TUBA1A/α-tubulin in the MDA-MB-231, HCC1937, and HCC1954 REX cell lines compared to the HCC1954, BT474, and MCF7 cell lines (FIG. 3C and FIG. 9A). However, the expression of TUBA1A/α-tubulin in all EVs appeared similar, with a reduction only observed in the EVs derived from the normal cell subtype Hs 371 T. Additionally, as shown in FIG. 3D and FIG. 9B, a slight correlation between TUBB3/βIII-tubulin expression and paclitaxel sensitivity in cells was observed, with a tendency of reduced expression of TUBB3/βIII-tubulin in the MDA-MB-231 and HCC1937 cell lines. Interestingly, comparable levels of TUBB3/βIII-tubulin were seen in EVs isolated from both HCC1954 parental and HCC1954 REPX. In case of the microtubule modulator, CIC-3, present results demonstrated reduced expression of CIC-3 in the MDA-MB-231, HCC1937, and HCC1954 REX cell lines compared to the HCC1954, BT474, and MCF7 cell lines (FIG. 3E). Unfortunately, the levels of CIC-3 in EVs were notably low, except in the EVs derived from HCC1954 REPX (FIG. 3E). These outcomes collectively suggest a potential differential modulation of various tubulin isoforms and the regulation of polymeric versus free forms. Consequently, these three biomarkers, TUBA1A/α-tubulin, TUBB3/βIII-tubulin, or CIC-3, may not be fully reliable indicators, given the intricacies and potential interplay with additional factors that could affect their accuracy.

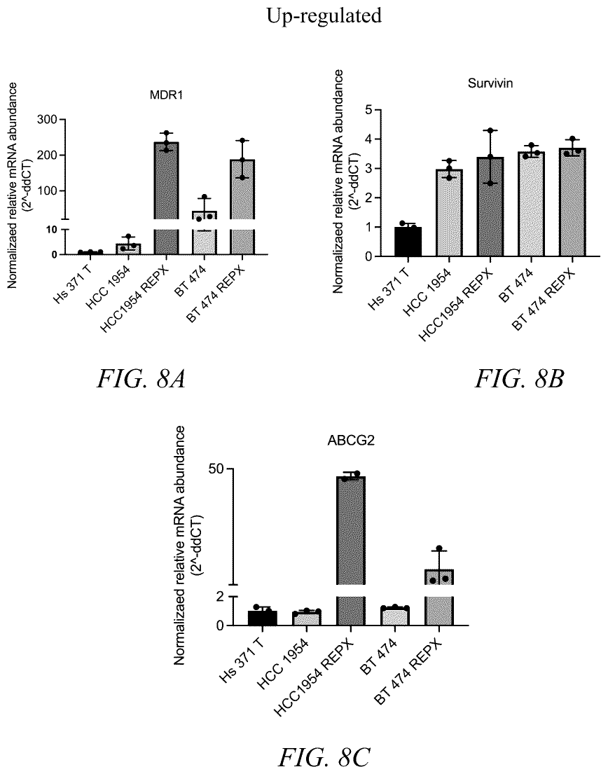

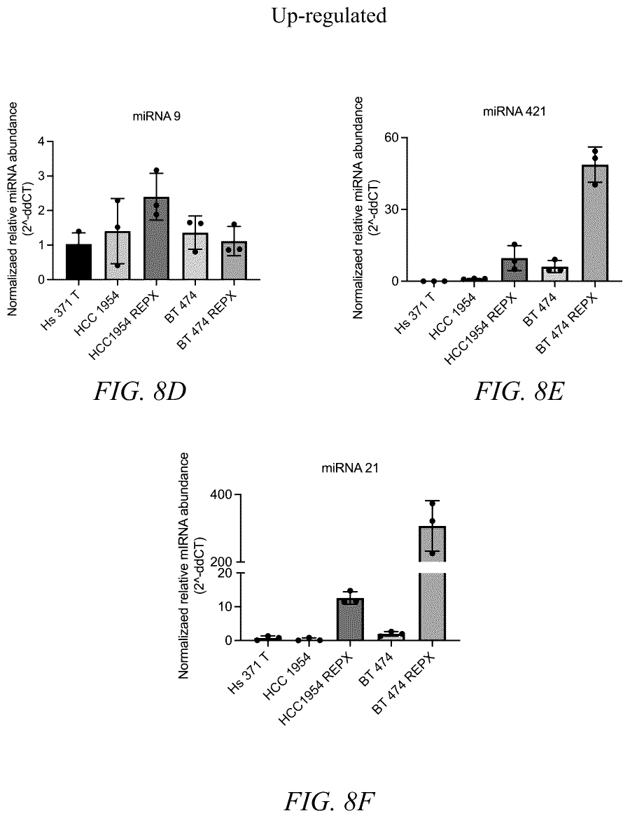

Survivin expression (FIG. 3F and FIG. 8B) exhibited a notable increase across the MDA-MB-231 and HCC1937 cell lines, and the paclitaxel-resistant HCC1954 PX, as well as in their corresponding EVs, in contrast to the more sensitive and normal cell lines and their corresponding EVs.

Genes regulating cell cycle and proliferation, such as CCND1/cyclin D1 and PTGES3, have been reported to be associated with the response to paclitaxel through their interaction with G2/M-associated events [Adekeye et al., 2022, 34,920,330; Bao et al., 2020, 32796817]. A reduction in CCND1/cyclin D1 mRNA levels (FIG. 3G and FIG. 9D) was observed in the MDA-MB-231 and HCC1937 cell lines compared to the HCC1954, BT474, and MCF7 cell lines, except in HCC1954 REPX cells. Similar trends were observed in their corresponding EVs (FIG. 3G). PTGES3 (FIG. 3H) exhibited a completely opposite expression in cell lines versus EVs. PTGES2 expression was lower in the less sensitive cell lines, MDA-MB-231, HCC1937, and HCC1954 REPX, compared to the more sensitive cell lines, HCC1954, BT474, and MCF7. However, in EVs derived from the HCC1954, BT474, and MCF7 cell lines, PTGES2 expression was increased when compared to EVs derived from the MDA-MB-231, HCC1937, and HCC1954 REPX cell models (FIG. 3H).

In case of the miRNA molecules, miR-421 (FIG. 3I and FIG. 8E) and miR-9 (FIG. 3J and FIG. 8D) exhibited higher expression in the MDA-MB-231, HCC1937, and HCC1954 REX cell lines, but these miRNAs were not detected using the same technique in their corresponding EVs. Only miR-21 (FIG. 3K and FIG. 8F) could be detected and analyzed at both cell and EV levels. In cells and EVs, an increase in expression was observed in the less sensitive and resistant models, MDA-MB-231, HCC1937, and HCC1954 REPX, compared to the more sensitive breast cancer and normal models, HCC1954, BT474, MCF7, and Hs 371 T.

### Example 4. P-gp, Survivin, and Cyclin D1 Protein Expression in Cell Lines and EVs

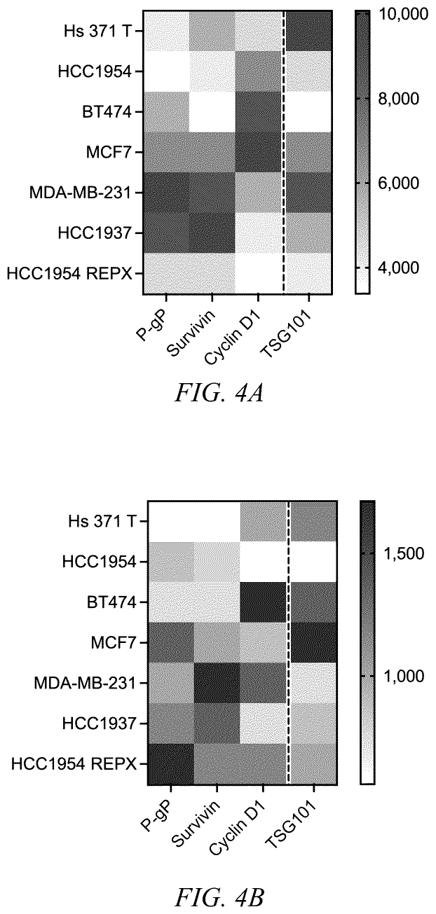

Following the qPCR results, the protein expression of P-gp, survivin, and cyclin D1 was assessed in breast cancer cell models HCC1954, BT474, MCF7, MDA-MB231, HCC1937, and HCC1954 and a normal cell line (Hs 371 T; FIG. 4A), along with their respective EVs (FIG. 4B). TSG-101 served as the internal control.

The data revealed a correlation between P-gp and survivin protein levels and paclitaxel sensitivity (FIGS. 4A-4B). The protein concentration of both biomarkers was higher in the less sensitive cells (MDA-MB-231 and HCC1937) compared to the levels observed in the more sensitive cell lines (HCC1954, BT474, and MCF7) and the normal cell line (Hs 371 T). Notably, although the expression of these two proteins was lower in HCC1954 REPX compared to the less sensitive cell models, it remained higher than the expression in the parental cell subtype (HCC1954). The protein profiles of P-gp and survivin in EVs mirrored their expression in cells.

Cyclin D1 protein expression was reduced in the less sensitive cell lines, (FIG. 3G and FIG. 9D). However, the protein levels in EVs did not show a clear correlation with paclitaxel sensitivity.

### Example 5. Single EV Analysis for P-Gp and Survivin on NPOP Substrate Using Double Labeling of EV and Biomarkers

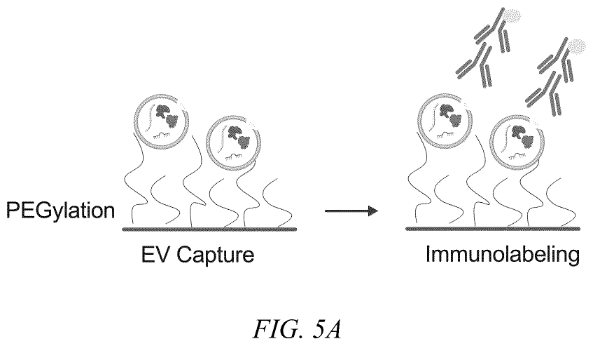

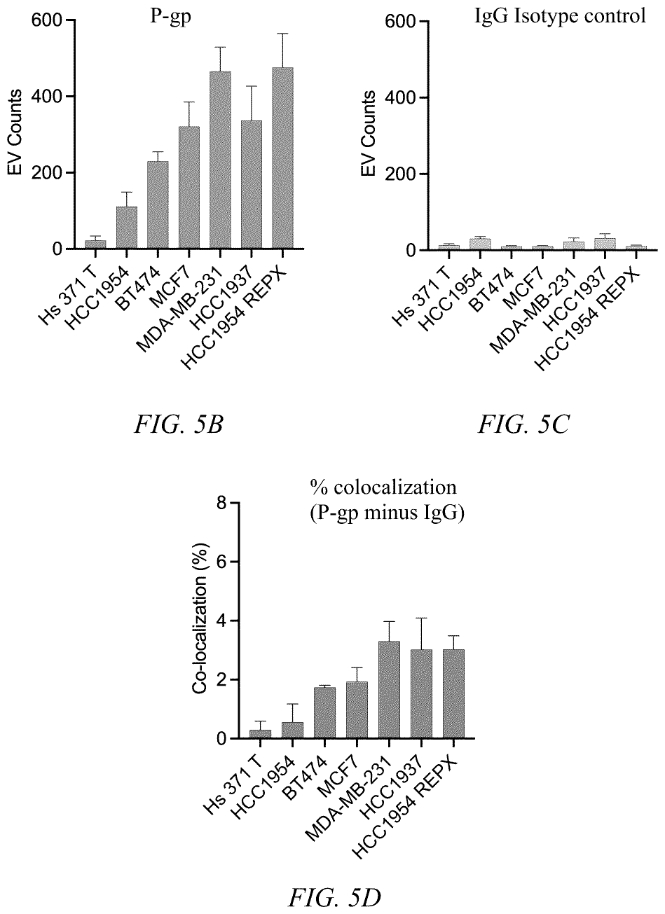

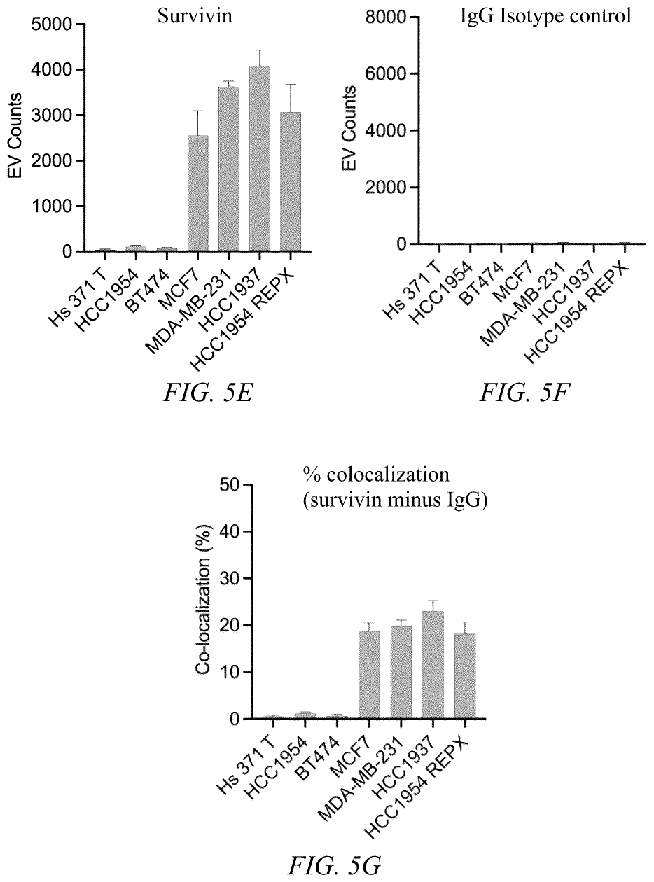

Further exploration was conducted into P-gp and survivin expression at the individual EV level. The molecular profiling capabilities of the NPOP substrate for P-gp and survivin were assessed in six breast cancer cell-derived EVs (HCC1954, BT474, MCF7, MDA-MB-231, HCC1937, and HCC1954 REPX) and one normal cell-derived EV (Hs 372 T). Initially, cell line-derived EVs (FIG. 5A) were labeled with Alexa Fluor™ 555 (AF555) and attached to the functionalized substrate using the SH-PEG-COOH linker. Subsequent immunofluorescence staining was conducted using optimized concentrations of respective antibodies against P-gp (FIGS. 5B-5D) or survivin (FIGS. 5E-G), followed by labeling with Alexa Fluor™ 488 or Alexa Fluor™ 647 dye. The count of Alexa Fluor™ 488-labeled EVs (biomarker channel—P-gp) or Alexa Fluor™ 647-labeled EVs (biomarker channel—survivin) showing co-localization with the Alexa Fluor™ 555 signal (EV channel) was analyzed and converted into co-localization percentage values. As a control, Isotype labeling was used.

Both P-gp and survivin exhibited high colocalization with EV marker in the EV samples derived from the MDA-MB-231, HCC1937, and HCC1954 REX cell lines compared to the HCC1954, BT474, and MCF7 cell lines. (FIGS. 4B-4G and FIGS. 10D-10E). The isotypes control showed only a few instances of co-localization across all cell line-derived EVs. These findings reaffirm that the presence of EVs positive for P-gp and survivin strongly correlates with the acquisition of paclitaxel resistance.

### Example 6. Longitudinal Monitoring of Paclitaxel Response in Breast Cancer Patients: Proof-of-Principle Testing

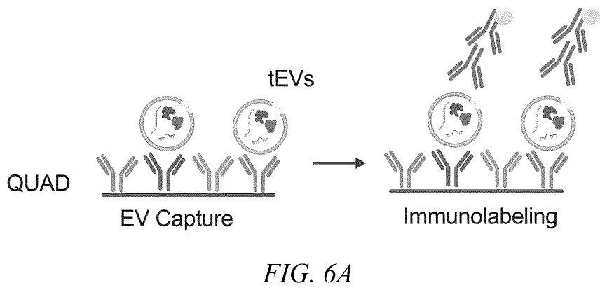

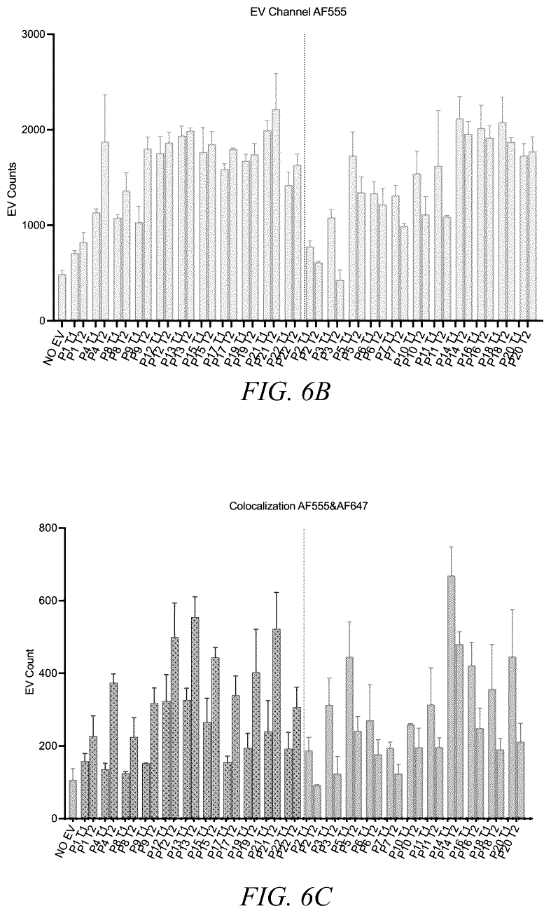

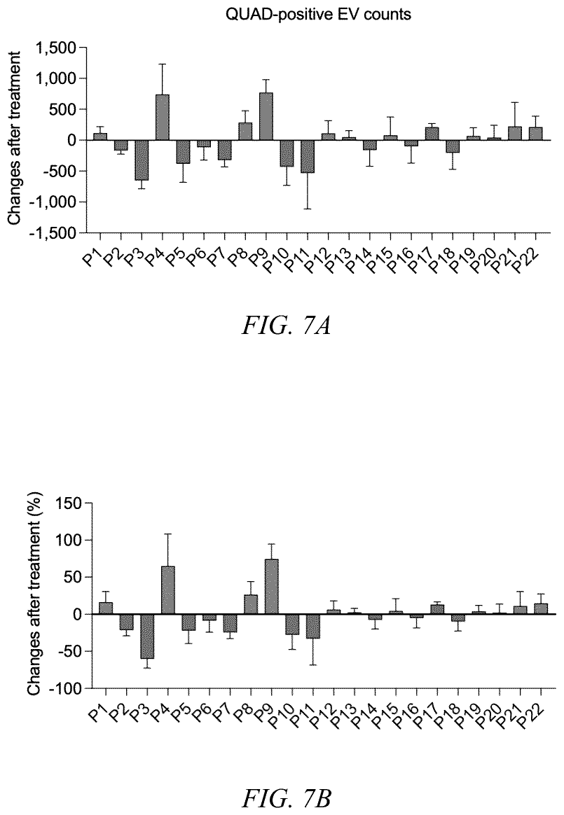

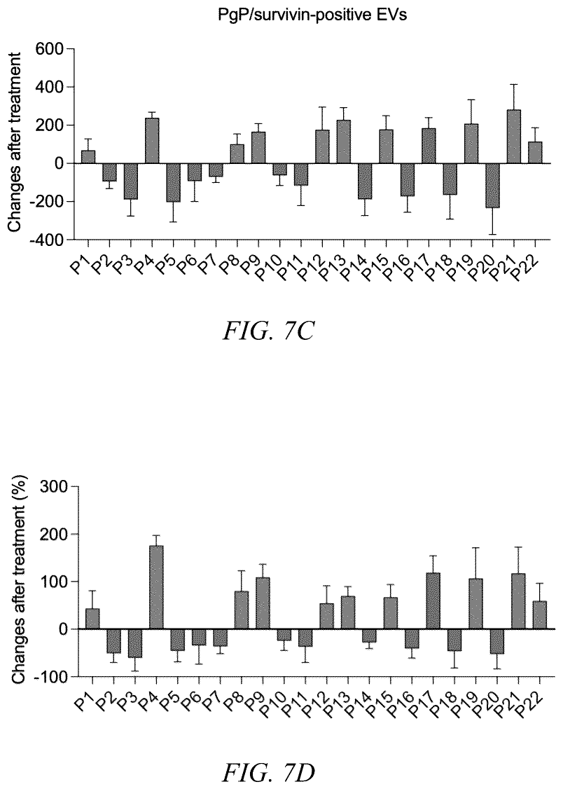

In breast cancer, the QUAD markers (MUC1, HER2, EGFR, and EpCAM) are helpful for identifying and isolating tumor-derived extracellular vesicles (tEVs), offering a potential method to detect and study cancer-associated EVs circulating in the body. Employing a multiplexing approach, the discriminatory power of the QUAD marker mix was combined to selectively differentiate tEVs from normal host-EVs with the quantification of colocalization of P-gp and survivin to predict paclitaxel response (FIG. 6A).

In a pilot study, 44 patient samples (n=22) were analyzed. Two plasma samples were collected per patient: before chemotherapy (T1) and after neoadjuvant chemotherapy (T2). The clinical details of these patients are described in Table 4.

Within this cohort, an increase in colocalization counts visualized by Alexa Fluor™ 555-labeled QUAD marker antibody mix and Alexa Fluor™ 647-labeled P-gp/survivin antibody mix was identified in T2 compared to T1 among 11 patients (FIGS. 6B-6C), classified as poor responders. Conversely, the remaining 11 patients were classified as good responders due to reduced co-colocalized EVs in T2 compared to T1. Among these 22 patients, and based on the clinical outcome, 21 out of 22 patients (a success rate of over 95%, FIG. 6C) were successfully identified as a paclitaxel responder or paclitaxel non-responder based on QUAD markers plus P-gp/survivin biomarker colocalization.

TABLE 4
Clinical information of patients

| | pCR | no pCR |
|---|---|---|
| | [n = 12] | [n = 10] |
| Age [yr.] | 50.5 | 49.5 |
| Median | [36-73] | [36-65] |
| BMI | 23.7 | 23.0 |
| Median [range] | [19.4-27.3] | [18.0-32.4] |
| Lesion size on imaging [mm] | 20.5 | 25.0 |
| Median [range] | [7-50] | [13-50] |
| CT * | | |
| 1 | 5 | 3 |
| 2 | 6 | 6 |
| 3 | 1 | 1 |
| CN * | | |
| 0 | 6 | 5 |
| 1 | 6 | 3 |
| 2 | 0 | 0 |
| 3 | 0 | 1 |
| Histological type at core biopsy * | | |
| Ductal | 12 | 10 |
| Lobular | 0 | 0 |
| Grading at core biopsy * | | |
| 1 | 0 | 0 |
| 2 | 2 | 3 |
| 3 | 10 | 7 |
| Pgr * | | |
| negative | 10 | 7 |
| positive | 2 | 3 |
| Er * | | |
| negative | 5 | 3 |
| positive | 7 | 7 |
| c-erbB2 * | | |
| negative | 4 | 2 |
| positive | 8 | 8 |
| Ki67 at core biopsy * | | |
| 0-20 | 4 | 4 |
| >20 | 8 | 6 |
| Neoadjuvant chemotherapy regimen * | | |
| Anthracycline/FEC + Taxanes | 4 | 2 |
| Anthracycline/FEC + | 8 | 8 |
| Taxanes + anti-HER2 | | |

* Where not specified the number of subjects is reported.
cN: Pre-treatment clinical N stage;
cT: Pre-treatment clinical T stage;
Pgr: Progesterone receptor;
Er: Estrogen receptors;
c-erbB2: human epidermal growth factor receptor 2.

### REFERENCES

1. Fulwah Y. Alqahtani et al. Prof. of Drug Substances. Excipients and Related Methodology. 44, 205 (2019).
2. Nemcová-Fürstová et al. Toxicol. Appl. Pharmacol., 310, 215 (2016)
3. Kreger et al. Cancers. 8, 111 (2016)
4. George Dura et al. British Journal of Cancer. 116, 1318 (2017)
5. Francesca Montalto et al. Cells. 9, 2648 (2020)
6. Fu-Gang Duan et al. Cell Death and Disease, 10, 821 (2019)
7. Shi-Yun Cu et al. Cell. Mol. 17, 1207 (2013)
8. Duygu Selcuklu et al. Journal of Biological chemistry. 287, 29516 (2012)
9. Mi Ho Jeon et al. Adv. Sci. 10, 2205148 (2023)
10. Jae-Sang Hong et al. Adv. Sci. 2301766 (2023).
11. Mingyao Huang et al. Cancer Lett. 501, 234 (2021)

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for predicting chemotherapy resistance in a subject with cancer, the method comprising:
(a) providing a sample from the subject:
(b) isolating, detecting, or enriching tumor-derived extracellular vesicles (tEVs) from the sample, preferably wherein the tEVs are labeled with antibodies or antigen binding portions thereof that bind to tumor marker(s) and antibodies or antigen binding portions thereof that bind to chemotherapy resistance biomarker(s):
(c) determining the counts of tEVs positive for tumor marker(s) and chemotherapy resistance biomarker(s), or determining the intensity levels of tumor marker(s) and chemotherapy resistance biomarker(s) expressed in tEVs; and
(d) comparing the counts of tEVs or the marker intensity levels determined in step (c) to a reference level that represents the subject's cancer response to a chemotherapy, wherein the counts of tEVs or the marker intensity levels determined in step (c) that differ from the reference levels indicate whether the subject's cancer is resistant or sensitive to the chemotherapy.

2. The method of claim 1, further comprising using a plasmon-enhanced EV detection method in step (c).

3. The method of claim 1 or 2, wherein the antibodies or antigen binding portions thereof that bind to the EV tumor marker(s) further comprise fluorescent dyes.

4. The method of any one of claims 1-3, wherein the antibodies or antigen binding portions thereof that bind to the EV tumor markers comprise one or more antibodies or antigen binding portions thereof that bind to EpCAM, EGFR, MUC1, and/or HER2.

5. The method of any one of claims 1-4, wherein the EVs are detected using a protein-reactive TFP dye, wherein the TFP dye comprises fluorescent dye.

6. The method of claim 1, wherein the chemotherapy resistance biomarkers comprise protein or RNA.

7. The method of claim 6, wherein the chemotherapy resistance biomarkers are P-gp and survivin.

8. The method of any one of claims 1-7, wherein the quantification of EV tumor markers and chemotherapy resistance biomarkers comprises expression, concentration, intensity, or colocalization.

9. The method of claim 8, wherein the quantification of expression, concentration, intensity, or colocalization are analyzed using multichannel fluorescence imaging in a single EV.

10. The method of claim 1, wherein the cancer comprises breast cancer, ovarian cancer, and non-small cell lung cancer.

11. The method of claim 1 or 2, wherein the sample comprises tumor cells or plasma.

12. A method for monitoring drug-resistance longitudinally in a subject having cancer, the method comprising:
(a) providing a sample from the subject, where the sample is acquired from the same subject at multiple time points during treatment with a chemotherapy;
(b) isolating tumor-derived EVs (tEVs) from the sample, wherein the tEVs are further double labeled with EV tumor marker(s) and drug-resistance biomarker(s);
(c) determining colocalization of EV tumor marker(s) and drug-resistance biomarker(s); and
(d) detecting changes in colocalization of EV tumor marker(s) and drug-resistance biomarker(s) before and after the chemotherapy treatment, thereby determining drug-resistance in the subject based on the changes of the quantitative colocalization of EV marker(s) and drug-resistance biomarker(s) before and after chemotherapy treatment.

13. A method for monitoring drug-resistance in a subject with cancer over time, the method comprising:
(a) isolating tumor extracellular vesicles (tEVs) in a first sample obtained from a subject at a first time point, wherein isolating tEVs comprises:
(i) applying the first sample to a functionalized substrate to capture extracellular vesicles (EVs);
(ii) labeling the EVs with an antibody or antigen binding portions thereof that bind to a previously selected EV tumor marker(s); and
(iii) labeling the EVs with an antibody or antigen binding portions thereof that bind to previously a selected drug-resistance biomarker(s);
(b) determining a count of tEVs positive for tumor marker(s) and chemotherapy resistance biomarker(s) or a level of drug-resistance biomarker(s) in the tEVs at a first time point:
(c) administering one or more doses of a chemotherapy drug:
(d) isolating tEVs in a second sample from the subject obtained at a second time point, wherein the isolating tEVs comprises:
(i) applying the second sample to a functionalized substrate to capture EVs;
(ii) labeling the EVs with an antibody or antigen binding portions thereof that bind to the previously selected EV tumor marker(s) of step (a); and
(iii) labeling the EVs with an antibody or antigen binding portions thereof that bind to previously a selected drug-resistance biomarker(s) of step (a):
(e) determining the count of tEVs positive for tumor marker(s) and chemotherapy resistance biomarker(s) or the level of drug-resistance biomarker(s) in the tEVs at a second time point; and
(f) administering one or more additional doses of the chemotherapy drug to the subject if the count of tEVs positive for tumor marker(s) and chemotherapy resistance biomarker(s) or the relative level of drug-resistance biomarker(s) have not increased from the first time point to the second time point.

14. The method of claim 12 or 13 further comprising using a plasmon-enhanced EV detection method.

15. The method of any one of claims 12-14, wherein the tEVs are selected by a marker panel comprising EpCAM, EGFR, MUC1, and/or HER2.

16. The method of any one of claims 12-15, wherein the antibodies or antigen binding portions thereof that bind to EV tumor markers further comprise fluorescent dyes.

17. The method of any one of claims 12-16, wherein the drug-resistance biomarkers comprise protein or RNA.

18. The method of any one of claims 12-17, wherein the drug-resistance biomarkers are P-gp and survivin.

19. The method of any one of claims 12-18, wherein the quantification of the colocalization of EV marker(s) and drug-resistance biomarker(s) are analyzed using multichannel fluorescence imaging in a single EV.

20. The method of any one of claims 12-19, wherein the cancer comprises breast cancer, ovarian cancer, or non-small cell lung cancer.

21. The method of any one of claims 12-20, wherein the sample comprises plasma.

22. The method of any one of claims 12-21, wherein the method can identify drug-resistance prior to an observable increase in size of the subject's tumor.

23. The method of any one of claims 12-22, further comprising recommending, prescribing and/or administering a therapeutically effective amount of a chemotherapy to a subject.

24. A method monitoring drug-resistance in a subject with cancer over time, the method comprising:
(a) isolating tumor extracellular vesicles (tEVs) in a first sample obtained from a subject at a first time point, wherein isolating tEVs comprises applying the sample to a surface comprising capture antibodies or antigen binding portions thereof that bind to previously selected EV tumor marker(s) and labeling the captured tEVs with antibodies or antigen binding portions thereof that bind to previously a selected drug-resistance biomarker(s);
(b) determining a count of tEVs positive for tumor marker(s) and chemotherapy resistance biomarker(s) or a level of drug-resistance biomarker(s) in the tEVs at a first time point:
(c) administering one or more doses of a chemotherapy drug:
(d) isolating tEVs in a second sample from the subject obtained at a second time point after administration of the one or more doses of the chemotherapy drug, wherein isolating the tEVs comprises applying a second sample to a surface comprising capture antibodies or antigen binding portions thereof that bind to the previously selected EV tumor marker(s) from step (a) and labeling the captured tEVs with the antibodies or antigen binding portions thereof that bind to previously a selected drug-resistance biomarker(s) of step (a);
(e) determining a count of tEVs positive for tumor marker(s) and chemotherapy resistance biomarker(s) or a level of drug-resistance biomarker(s) in the tEVs at the second time point; and
(f) administering one or more additional doses of the chemotherapy drug to the subject if the count of tEVs positive for tumor marker(s) and chemotherapy resistance biomarker(s) or the relative level of drug-resistance biomarker(s) have not increased from the first time point to the second time point.

25. The method of claim 24, wherein the capture antibodies or antigen binding portions thereof that bind to the EV tumor markers comprise one or more antibodies or antigen binding portions thereof that bind to EpCAM, EGFR, MUC1, and/or HER2.

26. The method of claim 24 or 25, wherein the drug-resistance biomarker(s) comprises P-gp and/or survivin.

27. The method of any one of claims 24-26, wherein the cancer is breast cancer.

28. The method of any one of claims 1-27, wherein the chemotherapy is paclitaxel.

29. The method of any one of claims 24-28, wherein the first sample and/or the second sample comprises plasma.

30. The method of any one of claims 1-29, wherein the efficacy of predicting chemotherapy resistance over time in a subject with cancer is at least 95%.
